(19) United States
(12) Patent Application Publication
(10) Pub. No.: US 2010/0091244 A1
(43) Pub. Date: Apr. 15, 2010

(54) REAL IMAGE FORMING EYE EXAMINATION LENS UTILIZING TWO REFLECTING SURFACES WITH NON-MIRRORED CENTRAL VIEWING AREA

(76) Inventor: Donald A. Volk, Honolulu, HI (US)

Correspondence Address: ULMER & BERNE LLP;ATTN: DIANE BELL, 600 VINE STREET, SUITE 2800, CINCINNATI, OH, 45202, US

(21) Appl. No.: 12/505,270

(22) Filed: Jul. 17, 2009

Related U.S. Application Data
(60) Provisional application No. 61/135,455, filed on Jul. 19, 2008.

(51) Int. Cl. A61B 3/125; B29D 11/00

(52) U.S. Cl. 351/219; 264/1.1

(57) ABSTRACT

An inverted real image forming opthalmoscopic contact lens provides for viewing and treating structures within an eye. The lens comprises a contacting surface adapted for placement on the cornea of the eye, a concave annular anterior reflecting surface, a convex annular posterior reflecting surface, and two non-reflective portions. A first non-reflective portion is positioned along the lens axis and proximate to the convex annular posterior reflecting surface. A second non-reflective portion is positioned along the lens axis and proximate to the concave annular anterior reflecting surface. A light beam emanating from the structure of the eye enters the lens and contributes to the formation of an inverted real image of the structure through an ordered sequence of reflections of the light beam, first in a posterior direction from the anterior concave reflecting surface and next as a negative reflection in an anterior direction from the convex posterior reflecting surface.

### PRIORITY CLAIM

This application claims priority to, and the full benefit of, U.S. Provisional Patent Application No. 61/135,455, titled “REAL IMAGE FORMING EYE EXAMINATION LENS UTILIZING TWO REFLECTING SURFACES WITH NON-MIRRORED CENTRAL VIEWING AREA” and filed Jul. 19, 2008, which is incorporated by reference in its entirety.

### BACKGROUND OF THE INVENTION

1. Field of the Invention

The lens of the present disclosure relates to opthalmoscopic lenses for use with the slit lamp or other biomicroscope. More particularly the invention relates to diagnostic and therapeutic gonioscopic and indirect opthalmoscopic contact lenses that incorporate two annular reflecting surfaces which combine to provide positive power contributing to the formation of an inverted real image of the examined structures of the eye within the lens or element of the lens while optimally directing the light rays proceeding from the inverted real image to the objective lens of the biomicroscope for stereoscopic viewing and image scanning. The lens may be designed with a clear central viewing portion that facilitates positioning of the lens on an examined eye and allows direct viewing and treatment of other structures of the eye.

2. Description of Prior Art

Eye examination lenses including indirect and direct opthalmoscopy and gonioscopy lenses are used by ophthalmologists and optometrists for the diagnosis and treatment of the internal structures of the eye in conjunction with a slit lamp or other biomicroscope. Indirect opthalmoscopy lenses, such as the Volk 90D lens, generally comprise a single lens with two refracting surfaces that combine to provide positive power contributing to the formation of a real image of the patient's eye fundus anterior of the examined eye. Direct opthalmoscopy lenses, such as the Hruby lens, use minus power to produce a virtual image of the patient's eye fundus generally posterior of the examination lens. Some indirect and direct opthalmoscopic lenses are pre-set or hand held in front of the patient's eye while others incorporate a contacting means and interface with the cornea and tear layer of the eye. An example of a contact indirect opthalmoscopy lens would be the Volk QuadrAspheric® lens and an example of a contact direct opthalmoscopy lens would be the Volk Centralis Direct® lens. Indirect opthalmoscopy lenses provide a wide field inverted view while direct opthalmoscopy lenses provide a small field with high magnification and high resolution in correct orientation.

Diagnostic lenses such as the Goldmann lens, Zeiss four mirror gonioscopy lens and Keoppe lens contact the eye and are used to examine and treat structures of the anterior chamber of the eye, specifically in the area of the anterior chamber angle, or iridocorneal angle. The four-mirror lens incorporates angulated mirrors and like the other gonioscopy lenses operates to eliminate the power of the cornea to avoid total internal reflection of the light rays at the cornea-air interface. Light rays from the anterior chamber angle enter the lens and are reflected by mirrors along the line of vision of the viewer, one for each quadrant of the examined eye. In that a single mirror is used for each of the four sectional views, each image is reverted and discontinuous with the other sectional views. Furthermore the field of view obtainable through each mirror is very small. The Goldmann lens performs in an identical manner to the Zeiss four mirror lens except that it has only a single mirror used for gonioscopy. The Keoppe lens employs a contact lens having a rather highly curved convex anterior surface and a thickness sufficient to prevent total internal reflection of incident light rays from the anterior chamber angle from its convex surface, thereby allowing light rays to pass through for examination purposes. There is no real conjugate pupil formed by the Keoppe lens and the physician may only obtain a small field of view at an extremely angled inclination relative to the eye axis through a stereoscopic viewer.

Real image forming ‘indirect opthalmoscopic’ viewing systems have also been suggested for viewing structures of the anterior chamber. A theoretical advantage of such a system lies in the continuous and uninterrupted 360 degree field of view that may be provided in the form of an annular section corresponding to the structures of the anterior chamber angle, viewed with the slit lamp biomicroscope in its normal orientation. Such a system is described in U.S. Pat. No. 6,164,779 to Volk (“the '779 patent”). This patent sets forth a series of lenses comprising a first corneal contacting lens system receiving light rays originating at the anterior chamber angle and a second imaging forming system receiving light rays from the first lens system producing a real image of the anterior chamber angle outside of the patient's eye. Various embodiments include refracting as well as reflecting surfaces providing positive power for focusing light rays. Although the '779 patent presents a theoretically plausible real image forming gonioscopy lens design, the complexity required of the majority of embodiments in order to provide a correctly oriented real image and to redirect highly angulated light rays proceeding from the intermediate image to the final correctly oriented image results in aberrations that precludes its use in diagnostic or treatment applications. Other less complex embodiments of the '779 patent employing fewer lens elements display either chromatic aberration, severe field curvature, low magnification and/or glaring reflections from central mirrored sections which obstruct light passage through the center of the lens, thus rendering the lens not useful.

U.S. Pat. No. 7,144,111 to Ross, III, et al. (“the '111 patent”), represents an attempt to provide an improved real image forming gonioscopy lens. Although achromatized and corrected for other aberrations, the lenses depicted in the embodiments of the '111 patent exhibit numerous disadvantages that preclude its successful application, including excessive weight, an excessive lens length of over 35 mm, an excessive distance from the examined eye to the image plane of over 51 mm, which is beyond the positioning range of the slit lamp biomicroscope, and poor stereoscopic visualization and image scanning capability resulting from the small light ray footprint at the biomicroscope objective lens aperture.

In co-pending U.S. patent application Ser. No. 12/229,747, titled Real Image Forming Eye Examination Lens Utilizing Two Reflecting Surfaces and filed on Aug. 25, 2008, an eye examination lens particularly well suited for gonioscopic examination of the eye is disclosed. The lens provides a continuous and uninterrupted annular field of view of the anterior chamber angle as an inverted image viewed stereoscopically with the slit lamp biomicroscope.

In co-pending U.S. patent application Ser. No. 12/321,709, titled Real Image Forming Eye Examination Lens Utilizing Two Reflecting Surfaces Providing Upright Image and filed on Jan. 22, 2009, another lens for gonioscopic examination of the eye is disclosed. The lens provides a continuous and uninterrupted annular field of view of the anterior chamber angle in upright and correct orientation, viewed stereoscopically with the slit lamp biomicroscope.

### SUMMARY OF THE INVENTION

Based on the foregoing there is found to be a need to provide a real image forming gonioscopy lens that avoids the problems associated with the prior art lenses and which in particular provides an inverted real image of the structures of the eye, has excellent optical attributes and good magnification properties, is easily positioned and manipulated within the orbital area of the examined eye, provides visualization through the center of the lens that facilitates its application to the eye and eliminates glaring reflections that are disturbing to the practitioner and handicap diagnosis and treatment procedures. It is therefore a main object of the invention to provide an improved diagnostic and therapeutic gonioscopy lens that incorporates two reflecting surfaces that combine to provide positive power contributing to the formation of a real image that is inverted with respect to the structures of the eye.

It is another object of the invention to provide a diagnostic and therapeutic gonioscopy lens that provides a continuous and uninterrupted annular field of view.

It is another object of the invention to provide a diagnostic and therapeutic gonioscopy lens that is well corrected for optical aberrations including astigmatic error, chromatic aberration, and field curvature.

It is another object of the invention to provide a diagnostic and therapeutic gonioscopy lens that comprises as few as one or two optical elements.

It is another object of the invention to provide a diagnostic and therapeutic indirect opthalmoscopy lens that incorporates two reflecting surfaces that combine to provide positive power contributing to the formation of a real image that is inverted with respect to the structures of the eye.

It is another object of the invention to provide a diagnostic and therapeutic indirect opthalmoscopy lens that provides a continuous and uninterrupted annular field of view of the mid-peripheral retina.

It is another object of the invention to provide a diagnostic and therapeutic gonioscopy or indirect opthalmoscopy contact lens that provides visualization of the patient's eye during its application to the cornea and allows diagnostic and treatment capabilities directly through a non-mirrored central refracting portion of the lens.

These and other objects and advantages are accomplished by a diagnostic and therapeutic eye examination lens that incorporates two reflecting surfaces that work in concert to provide positive power contributing to the formation of a real inverted image. The optical materials selected and curvatures provided result in a lens with improved optical quality, practicality of function and simplicity of design.

The lens of the present disclosure functions as both a condensing lens, directing light from the illumination portion of a biomicroscope to the visualized eye structures, and an image-forming lens, producing a real image of the illuminated eye structures in an image plane anterior of the examined eye. The light pathways through the lens are folded through the use of two reflecting surfaces that optimally correct optical aberrations while shortening the distance to the plane of the real image.

The term “opthalmoscopic contact lens” as used in this disclosure refers to a contact lens for diagnosis or laser treatment of the interior structures of the eye including those of the fundus within the posterior chamber and the iris and iridocorneal angle within the anterior chamber. The opthalmoscopic contact lenses described in this disclosure may be used for general diagnosis as well as for treatment by means of the delivery of laser energy to the trabecular meshwork and adjacent iris structures of the eye, i.e., laser trabeculoplasty, peripheral laser iridoplasty, laser iridotomy, or in the delivery of laser energy in the treatment of the equatorial and peripheral retina. The lens of the present disclosure may also provide diagnostic or treatment capability of eye structures including the central retina, vitreous and lens capsule as a virtual image viewed centrally through only the refracting media of the lens, without the use of the mirror system. The clear central viewing portion may also facilitate positioning of the lens on an examined eye by allowing the practitioner to visualize the eye directly through the lens with the biomicroscope as it is brought into contact with the cornea.

In a first group of embodiments a light beam proceeding through the lens from the examined eye to the inverted real image is reflected in an ordered sequence of reflections first as a positive reflection in a posterior direction from a concave anterior reflecting surface and next as a negative reflection in an anterior direction from a convex posterior annular reflecting surface. In a second group of embodiments a light beam proceeding through the lens from the examined eye to the inverted real image is reflected in an ordered sequence of reflections first as a negative reflection in a posterior direction from a concave anterior reflecting surface and next as a negative reflection in an anterior direction from a convex posterior reflecting surface. In both groups of embodiments each of the anterior and posterior reflecting surfaces are formed as an annulus. In such embodiments, a first non-reflective portion can be positioned along the lens axis and proximate to the posterior reflecting surface, and a second non-reflective portion can be positioned along the lens axis and proximate to the anterior reflecting surface. Such an arrangement provides for transmission of light directly through the lens and may additionally prevent glaring slit lamp light source reflections from optical surfaces from interfering with diagnostic and treatment procedures, which can be disturbing to the practitioner.

A ‘positive reflection’ is defined as a reflected central light ray or light beam that proceeds from the point of reflection further from the axis of the lens than the incident ray as determined by the point of intersection of each with a perpendicular to the axis of the lens.

Conversely, a ‘negative reflection’ is defined as a reflected central light ray or light beam that proceeds from the point of reflection closer to the axis of the lens than the incident ray as determined by the point of intersection of each with a perpendicular to the axis of the lens.

The terms ‘lens axis’ and ‘axis of the lens’ refer to the theoretical line that passes through the centers of curvature of all optical surfaces of a lens including rotationally symmetric aspheric surfaces or an approximate physical center of a lens or lens system.

A ‘Y’ direction as used in this disclosure refers to the dimension or direction perpendicular to the axis of the lens.

A “Z” direction defines the dimension or direction along or parallel to the axis of the lens. ‘Z’ directionality on a lens layout is negative leftward of a Z zero reference point and positive rightward of the same Z zero reference point. All lenses in this disclosure are defined with the contacting surface in a leftward positioned, −Z position relative to the first reflecting surface, which is in a rightward positioned, +Z position relative to the contacting surface.

By ‘posterior direction’ is meant the −Z direction of reflection from a Z reference point located at the point of reflection

By ‘anterior direction’ is meant the +Z direction of reflection from a Z reference point located at the point of reflection.

By ‘light’ is meant electromagnetic radiation, both visible and invisible, including ultraviolet and infrared wavelengths.

By ‘light ray’ is meant an idealized line of light.

By ‘light beam’ is meant the parallel, convergent, or divergent light that initially emanates from a point of a structure of the eye and contributes to the formation of an image produced by a lens. The curvatures and limiting dimensions of the refracting and/or reflecting surfaces of the lenses of this disclosure affect the size and convergence or divergence of a light beam.

By ‘central ray’ is meant the light ray that is centrally positioned within a light beam as viewed in the Y,Z plane.

By ‘lenticulated surface’, ‘lenticulated design’ and ‘lenticular’ is meant a surface or surface design having discontinuous curvatures.

By ‘oblate’ is meant a curvature as least a portion of which has increasing curvature peripheralward.

By ‘convex’ or ‘partially convex’ is meant a surface curvature at least a portion of which either or both the sagittal and tangential radii define a convex curvature.

By ‘concave’ or ‘partially concave’ is meant a surface curvature at least a portion of which either or both the sagittal and tangential radii define a concave curvature.

By ‘internally reflecting’ is meant a reflection from the side of a mirror surface against the glass or plastic material to which it is applied.

By ‘externally reflecting’ is meant a reflection from the side of a mirror surface opposite the side of the glass or plastic material to which it is applied.

By ‘non-reflective’ is meant the quality of effectively being absent of reflection or essentially being absent of specular reflection, or being non-mirrored.

By ‘multi-element lens’ is meant a lens incorporating at least two elements interfaced together with a liquid, gel or optical cement.

By ‘non-transmissive’ is meant the quality of partially or fully not transmitting at least one wavelength of light.

By ‘vertex’ is meant the point of a surface or the curvature defining a surface through which the lens axis passes or which is the physical center of a surface or the curvature defining a surface.

In some embodiments a single element consisting of two reflecting and refracting surfaces may comprise the entire lens. In other embodiments additional lens elements may be incorporated to enhance the optical qualities of the lens.

The lens may be produced of either plastic material such as polymethylmethacrylate (pmma), polycarbonate, polystyrene, ally diglycol carbonate (CR-39®) or any other suitable polymeric material or any glass material, for example N-BK7 (available from Schott AG) and S-FPL51Y, S-LAH59 or S-LAH58 glasses (available from Ohara Corp). An optical material with a refractive index over 1.66, such as S-LAH58 glass, used in the lens element through which the light beam passes between the anterior and posterior reflecting surfaces, provides the benefit of bending rays entering that element in a direction towards the optical axis of the lens, thus reducing the distance from the axis that light rays hit the anterior reflector, and thereby reducing the maximum diameter of the anterior reflector and therefore the diameter of the lens overall. Glasses with refractive indices ranging from below Nd=1.5 to above Nd=1.9 or greater may be utilized. Optical materials with specific Abbe values may also be utilized. For example, to reduce chromatic aberration, an optical material with an Abbe value greater than 56, such as S-FPL51Y glass having an Abbe value of 81.14, may be utilized to reduce color dispersion.

In the lens of the present disclosure the surface that comprises the anterior reflector and the refracting portion it surrounds may comprise a surface of continuous curvature, wherein both the reflecting and refracting portions are defined by the same surface parameters as a single curvature. Alternatively, the reflecting and refracting portions may be defined by different surface parameters, joining tangentially and without discontinuity or with discontinuity as a lenticular surface. The anterior reflector surface may be concave with a spherical or aspheric contour, and if aspheric may comprise a polynomial-defined asphere at least a portion of which is concave. The refracting portion may be concave, plano or convex. The surface that comprises the posterior reflector and a refracting portion it surrounds may also be defined by different surface parameters, joining tangentially or forming a lenticular surface as above described. The posterior reflector may be convex with a spherical or aspheric contour, and if aspheric may comprise a polynomial-defined asphere at least a portion of which is convex. The inventor has discovered that the above first stated combination of reflections, in which the first reflection is a positive reflection in a posterior direction from the anterior annular reflecting surface and the following reflection is a negative reflection in an anterior direction from the posterior annular reflecting surface, may correct field curvature of the image to a high degree particularly when the angle of incidence and reflection of a central light ray reflecting from the posterior reflecting surface is very low, resulting in the central ray of a beam originating at the iridocorneal angle deviating from parallel to the lens axis preferably by less than 15° and more preferably by less than 8° after reflection from the posterior reflecting surface. The low deviation angle of the central ray further assists in directing the light beam from the inverted real image to the biomicroscope objective lens such that the span of the light beam at the biomicroscope objective lens covers the extent of the biomicroscope's left and right microscope lenses, thus insuring binocular and stereoscopic biomicroscope visualization of the inverted image. Furthermore, when the value of the combined angles of incidence or reflection for a central ray reflecting from both the anterior and posterior reflecting surfaces is maintained at a very low value, preferably below 24.5°, and more preferably below 18.5°, aberrations overall may be maintained at a minimum. The inventor has also discovered that a gonioscopy lens providing the second stated combination of reflections in which the first reflection is a negative reflection in a posterior direction from an anterior annular reflecting surface and the following reflection is a negative reflection in an anterior direction from a posterior annular reflecting surface provides an improved lens construction with added transmission of light directly through the lens for an enhanced diagnostic capability and/or the elimination of glaring reflection from a biomicroscope light source that interferes with clinical procedures and which is disturbing to the practitioner.

All refracting surfaces in the various embodiments disclosed other than the contacting surface adapted for placement on a cornea may be concave, convex, plano or defined as a polynomial surface having both concave and convex attributes, including the surface of a multi-element lens opposite the contacting surface in embodiments wherein a posterior refracting surface adjoins the posterior reflecting surface, thereby providing a contacting element that is bi-concave, plano-concave or meniscus in shape.

As an alternative to the use of optical cement as an interface medium in the various multiple element lens embodiments shown and described in this disclosure, gel and liquid interface mediums may be utilized instead, thus allowing separation of the component elements for sterilization purposes. A liquid or gel medium also provides a means to interface an intermediate or anterior glass reflecting element with a separate and disposable contacting portion comprising the contacting element and an open ended frustoconically shaped container for receiving the reflecting portion. The curvatures of two surfaces optically coupled at the interface of an optically coupled lens need not have exactly the same curvature and may have different curvatures.

Chromatic aberration of the lens of the present disclosure may be corrected to a high degree as the reflecting surfaces together provide significant positive power contributing to the formation of the inverted real image, thus allowing the refracting surfaces to be tailored to minimize or practically eliminate dispersion.

Scanning of the real image may be accomplished by lateral and vertical movement of the biomicroscope and in conjunction with angulation or tilting of the gonioscopy lens on the eye the visualized area may be expanded to include a larger extent of the iris and the inner corneal surface adjacent the iridocorneal angle.

Other features and advantages of the invention will become apparent from the following description of the invention in conjunction with the accompanying drawings.

### BRIEF DESCRIPTION OF THE DRAWINGS

FIG. 1 shows a lens layout and ray tracing of a two-element gonioscopy lens according to a first embodiment of the invention.

FIG. 2a shows a detailed view of the lens of FIG. 1.

FIG. 2b shows the central ray pathway of a light beam shown in FIG. 2a.

FIG. 3 shows the lens of FIGS. 1 and 2 including light beam pathways pertaining to direct imaging of the iris through the center of the lens.

FIG. 4 shows a lens layout and ray tracing of a single element gonioscopy lens according to a second embodiment of the invention.

FIG. 5 shows a lens layout and ray tracing of a two-element gonioscopy lens according to a third embodiment of the invention.

FIG. 6 shows a lens layout and ray tracing of a two-element gonioscopy lens according to a fourth embodiment of the invention.

FIG. 7 shows a lens layout and ray tracing of a three-element gonioscopy lens according to a fifth embodiment of the invention.

FIG. 8 shows various illumination systems in conjunction with the lens shown in FIG. 7.

FIG. 9 shows a lens layout and ray tracing of a three-element gonioscopy lens according to a sixth embodiment of the invention.

FIG. 10 shows a second view of the lens of FIG. 9 including light beam pathways pertaining to direct imaging of the central retina through the center of the lens.

FIG. 11a shows a lens layout and ray tracing of a two-element gonioscopy lens according to a seventh embodiment of the invention.

FIG. 11b shows a second view of the lens of FIG. 11a including light beam pathways pertaining to positioning the lens on an eye.

FIG. 11c shows a third view of the lens of FIG. 11a including light beam pathways pertaining to direct imaging of the posterior capsule of an eye.

FIG. 12 shows a lens layout and ray tracing of a three-element gonioscopy lens according to an eighth embodiment of the invention.

FIG. 13 shows a lens layout and ray tracing of a two-element indirect opthalmoscopy contact fundus lens according to a ninth embodiment of the invention.

### DETAILED DESCRIPTION

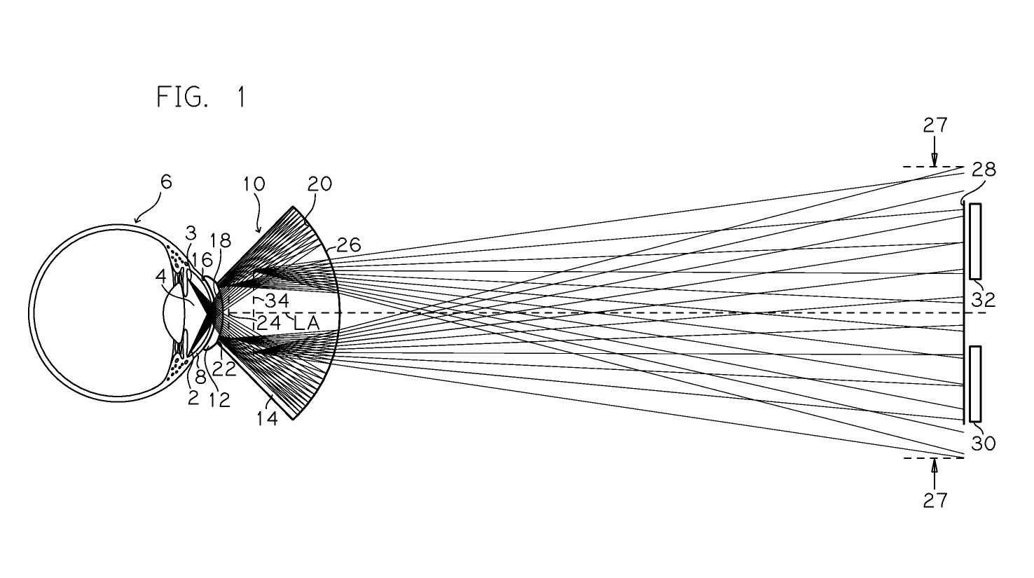

Referring to FIG. 1, there is shown a ray tracing and schematic cross-sectional view of an exemplary doublet gonioscopy lens according to a first embodiment of the invention, wherein lens 10 comprises an optically coupled lens including posterior contacting element 12 and anterior element 14. In this embodiment the anterior reflecting surface comprises an aspheric curvature and the posterior reflecting surface comprises a spherical curvature. Posterior element 12 is made of optical quality polymethylmethacrylate with an index of refraction of approximately Nd=1.492 and an Abbe number of approximately Vd=55.3, and anterior element 14 is made of S-LAH58 optical glass (available from Ohara Corp.) having an index of refraction of approximately Nd=1.883 and an Abbe number of approximately Vd=40.8. The two elements 12 and 14 are optically coupled at their interface using a suitable optical coupling material, including one of a variety of optical adhesives known to those skilled in the art, such as those available from Dymax Corporation and Norland Products. As a cemented doublet, the two elements 12 and 14 may be adhered together at their interface using optical adhesive 3-20261 manufactured by Dymax Corporation.

In practice the lens may be mounted in a holding frame or housing and applied to the cornea of a patient's eye in a manner similar to that used in conjunction with gonioscopic prisms and indirect opthalmoscopic contact lens and which is generally known to those skilled in the art. For ease of illustration the frame is not included in the present or subsequent figures. As previously mentioned an optically clear liquid or gel (such as saline or ophthalmic methylcellulose) may be utilized instead of an optical cement as the optical interface medium. As used in this disclosure the term ‘optically coupled’ describes doublet or triplet lenses in which the lens elements are optically coupled or interfaced with a liquid, gel or cement interface material and the term ‘interface’ describes such an optically coupled interface. A liquid or gel optical coupling medium allows separation of the component elements for sterilization purposes or alternatively provides a means to interface an intermediate or anterior glass reflecting element with a separate and disposable contacting portion incorporating the contacting element. A cement interface provides means to optically couple lens elements in a fixed relationship not requiring additional support to maintain the relative positions of the lens elements, whereas a lens having lens elements optically coupled with a liquid or gel material requires a means to maintain relative position and alignment between the coupled elements. Such a means to maintain relative position and lens element alignment may include a housing or holding frame as above mentioned formed as a frustoconically shaped container portion comprising the contacting element at its small end and an opening at the opposite larger end for receiving the anterior reflecting element. A small measured amount of saline, methylcellulose or other suitable liquid or gel optical interface material may be placed in the container portion on the surface of the contacting element opposite the contacting surface prior to the insertion of the anterior element. Once the anterior element is inserted into the container portion and brought into contact with the liquid or gel material, the liquid or gel material will be made to conform to both interface surfaces it contacts, and to form a thin section as it seeps between the surfaces. An optical cement, or liquid or gel interface coupling medium used in conjunction with an appropriately designed housing as described, may be utilized in the present and subsequent exemplary lenses and lens embodiments where an optical interface is indicated.

For illustrative purposes, only two light beams are shown emanating from point sources on opposite sides of axis of the lens within the anterior chamber of the schematic eye. Light beam 2 emanates from an iridocorneal point source and light beam 3 emanates from a peripheral iris point source. For ease of illustration, the tear film of the eye is not shown in the present or subsequent figures. Referring to FIG. 1, light beams 2 and 3 emanating from the stated iridocorneal and peripheral iris locations of anterior chamber 4 of eye 6 pass through the cornea 8 and tear layer of the eye and enter posterior contacting element 12 of lens 10 through corneal contacting surface 16 and continue through interface 18 into anterior lens element 14 and to concave annular reflecting surface 20 from which they are first reflected as a positive reflection in a posterior direction. The convergent light beams proceed in their respective directions to convex annular reflecting surface 22 from which they are next reflected as a negative reflection in an anterior direction. Proceeding from reflecting surface 22 the light beams focus at dotted line 24, which represents the field location of the inverted real image. The inverted real image is the final real image produced by the lens, that is, it is not an intermediate real image produced by the lens. The divergent light beams continue from inverted image 24 in their respective directions towards surface 26 where they are refracted and exit the lens. The light beams proceed towards biomicroscope objective lens aperture 28 and enter left and right microscope lenses 30 and 32, respectively, of the observing stereomicroscope. The stereomicroscope is adjusted to focus at virtual image plane 34 and provide an inverted view of the observed structures of the eye.

As can be seen in FIG. 1, span 27 of light beams 2 and 3 at the plane of biomicroscope aperture 28 exceeds the extent of left and right microscope lenses 30 and 32, thus insuring binocular and stereoscopic biomicroscope visualization of the inverted image both when the biomicroscope is coaxial with the lens as shown and when the biomicroscope is moved off axis to scan or bring peripheral image points to a more central location of the visual field of the biomicroscope. Biomicroscope objective lens aperture 28 is positioned approximately 100 millimeters from virtual image 34, which is an average objective lens focal length for commercially available slit lamp biomicroscopes. Slit lamp objective lens focal lengths generally range between 90 and 120 millimeters with most being between 95 and 105 millimeters. Although it is preferred that beam span 27 of at least light beam 2 exceed the extent of left and right microscope lenses 30 and 32, this is not necessary to achieve binocular and stereoscopic visualization with a slit lamp biomicroscope. A less broad beam span may provide binocular and stereoscopic viewing particularly when off axis scanning is reduced in extent. A further reduced beam, spanning an extent covering approximately 80% of both left and right microscope lenses, will also provide binocular and stereoscopic viewing when the biomicroscope is aligned coaxial with the lens. Based on typical slit lamp left and right microscope lens diameters and separation distances, span 28 preferably will be at least 30 mm in extent, more preferably at least 36 mm in extent and most preferably will be over 40 mm in extent. Operating microscopes have a broader range of working distances, ranging from around 150 millimeters to 300 millimeters, with some being as long as 400 millimeters, thus a lens designed for use with the slit lamp biomicroscope will also work with an operating microscope or other biomicroscopes with similar objective lens focal lengths. Alternatively, the lens may be modified in design to optimize performance as desired with a specific biomicroscope. The corresponding light beam span of lenses depicted in subsequent figures and embodiments may be similarly designed to provide binocular and stereoscopic viewing as shown and described with reference to FIG. 1.

As an alternative to the standard slit lamp or operating biomicroscope, a CCD, CMOS or other sensor based camera system incorporating the lens may be focused at the plane of the virtual image, thus allowing the light rays of the formed image that are refocused on the CCD or CMOS sensor to be converted to an analog or digital signal and then converted to an image, series of images or continuous video sequence displayed on a video monitor in real time for immediate diagnostic applications or digitally stored for subsequent review, electronic transmission or other applications. A similar alternative application provides that a CCD, CMOS or other image sensor be placed at the image plane of the lens slightly modified in design and truncated at the anterior end, thus allowing the light rays of the formed image that are directly focused on the sensor in like manner to be converted to an analog or digital signal and converted to an image, series of images or continuous video sequence displayed on a video monitor in real time for immediate diagnostic applications or digitally stored for subsequent review, electronic transmission or other applications. Both of the above electronic imaging systems may be utilized in conjunction with the lens of the present disclosure including that of the present embodiment as well as those of subsequent embodiments.

Illumination of the anterior chamber structures may be provided by the slit lamp biomicroscope's illumination system in a typical manner. The par focal illumination system will provide light to the anterior chamber following similar light pathways as shown, from the image plane back through the lens and cornea to the anterior chamber. Alternatively, illumination may be provided through optical fibers or through the use of an array of LED or OLED lamps positioned adjacent or within refracting surface 26 the emitted light of which is directed to pass through interface 18, cornea 8 and to the iris and iridocorneal angle, following similar but oppositely directed pathways to the rays emanating from the anterior chamber structures and proceeding to the first mirror surface, thereby illuminating selectively a portion of the anterior chamber or the entire circumference of the anterior chamber. Alternatively the optical fibers or LED's may direct their illumination along the outside of frustoconically shaped lens element 14 to or through contacting element 12 or directly to the cornea 8 of eye 6, thereby providing illumination of the anterior chamber without passing the illumination light rays through the lens. A further arrangement provides extremely small LEDs embedded in the portion of the lens adjacent the cornea, directing emitted light through the contacting surface to illuminate the structures of the anterior chamber. The above described fiber optic and LED illumination systems may provide continuous illumination of the illuminated structures even with movement of the biomicroscope during image scanning, and further may limit the amount of light passing through the pupil that illuminates the retina, thereby reducing patient discomfort and glaring slit lamp light source reflections from optical surfaces that interfere with diagnostic and treatment procedures and which are disturbing to the practitioner. The illumination systems may be affixed to or detachably removable from the opthalmoscopic contact lens and may be utilized in conjunction with the lens of the present embodiment as well as those of subsequent embodiments.

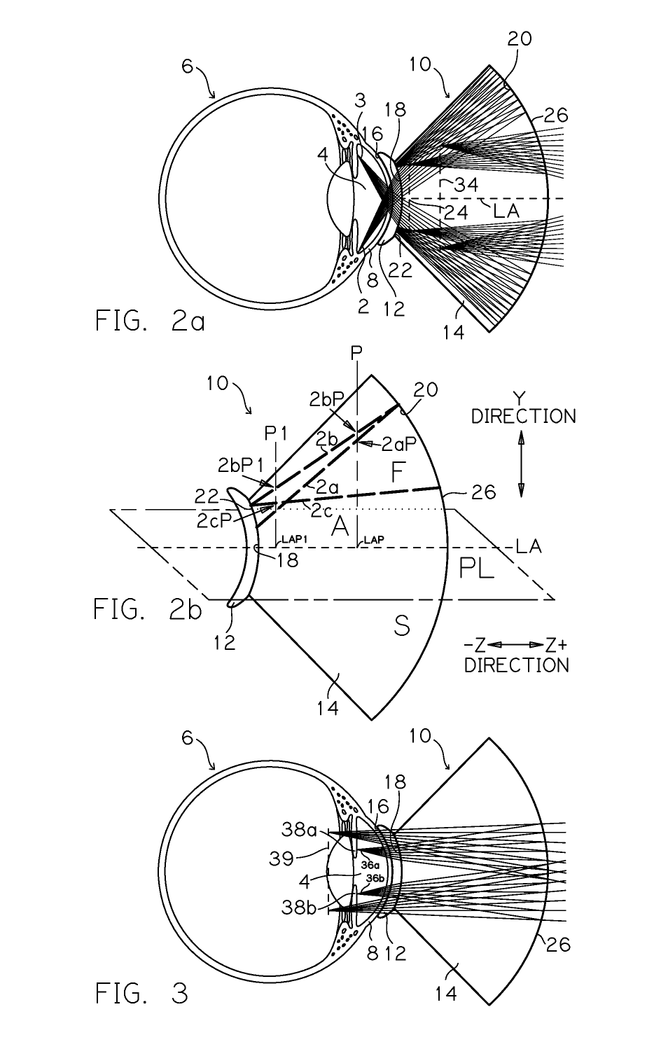

FIG. 2a shows the same lens as in FIG. 1 minus the diverging rays proceeding from the lens to the plane of the biomicroscope in order to better illustrate the light beam pathways and lens elements and surfaces. As previously described, light beams 2 and 3 emanating from the stated iridocorneal and peripheral iris locations of anterior chamber 4 of eye 6 pass through the cornea 8 and tear layer of the eye and enter posterior contacting element 12 of lens 10 through corneal contacting surface 16 and continue through interface 18, comprised of the anterior and posterior surfaces of lens elements 12 and 14 respectively, optically coupled with an interface material. As the light beams enter element 14 they are bent towards the axis of the lens LA due to the high refractive index of the glass comprising element 14, thereby reducing the outside diameter required of concave annular reflecting surface 20 from which each light beam is first reflected as a positive reflection in a posterior direction. The convergent light beams proceed in their respective directions to convex annular reflecting surface 22 from which each light beam is next reflected as a negative reflection in an anterior direction. Proceeding from reflecting surface 22 the light beams focus at dotted line 24, which as stated represents the field location of the inverted real image. The divergent light beams continue from inverted image 24 in their respective directions towards surface 26 where they are refracted and exit the lens. Virtual image 34, which is the apparent location of real image 24, is located 12.15 mm posterior of surface 26.

Contacting surface 16 comprises a concave surface adapted for placement on the patient's cornea, and may have a spherical or aspherical curvature. In the exemplary lens of this embodiment surface 16 has a radius of 8.0 mm and is spherical. Optical interface 18 is the interface of the central refracting portions of the anterior and posterior surfaces respectively of lens elements 12 and 14. The optical coupling material used to optically couple the interface surfaces may be used advantageously to fill gaps, variable distances or mismatches between the two interface curvatures. The curvature of interface 18 with respect to lens element 14 is spherical and concave with a radius of 10.97 mm. Reflecting surface 20 has an aspheric concave curvature with an apical radius of 12.45 mm and in combination with refracting surface 26 comprises a continuous aspheric curvature as the anterior surface of lens element 14. Reflecting surface 20 comprises an internally reflecting mirror-coated annular section having a 16 mm inner diameter that surrounds refracting surface area 26. Annular reflecting surface 22 has a convex spherical curvature with a radius of 10.97 mm, and in combination with the curvature of optical interface 18 comprises a continuous surface as the posterior surface of lens element 14. Reflecting surface 22 also comprises an internally reflecting mirror-coated annular section having a 6.3 mm inner diameter that surrounds optical interface 18, and is contained and protected within the lens at interface 18. Alternatively, annular reflecting surface 22 may comprise an externally reflecting section on the convex anterior surface of lens element 12, in which case reflected light beams proceeding from anterior reflecting surface 20 will pass through optical interface 18 prior to reaching posterior reflecting surface 22, and after reflection from posterior reflecting surface 22 will pass through optical interface 18 prior to entering lens element 14. The reflective sections may be mirrored by means of vacuum deposition of an evaporated or sputtered metal such as aluminum or silver, and protectively overcoated with a hardcoating, polymer or paint layer, as is known to those skilled in the art.

The lens of FIG. 2a may be modified to incorporate an anterior surface comprised of reflecting and refracting portions defined by different surface parameters, joining tangentially and without discontinuity, as previously mentioned. By so designing the anterior surface of the lens, parameters for the separate reflecting and refracting portions may be easily optimized. Modified surface radii for an exemplary lens having a slightly lower magnification include optical interface 18 having a radius of 11.92 mm, anterior reflecting surface 20 having an apical radius of 22.14 mm, posterior reflecting surface 22 having a radius of 11.92 mm and refracting surface 26 having an apical radius of 20.49 mm. Surfaces 20 and 26 join tangentially at a 16 mm diameter, which is the aperture value of refracting surface 26 and the inner diameter of annular mirror section 20. Lenses depicted in subsequent figures and embodiments may be similarly designed to incorporate surfaces comprising two portions that join tangentially and without discontinuity.

The exemplary lenses as shown and described with reference to FIG. 2a, each comprising a first anterior plus powered aspheric reflector paired with a second posterior minus powered annular spherical reflector, each which respectively produce the stated posterior and positive and anterior and negative reflections, provide a two-element optical system for a diagnostic and therapeutic gonioscopy lens with excellent imaging qualities that exhibits simplicity of design and ease of manufacture, utilizing two continuous surfaces that have both reflecting and refracting functions.

The formula

$$z = \frac{{cr}^{2}}{1 + \sqrt{1 - ( 1 + k ) {c}^{2} {r}^{2}}} + {a}_{1} r + {a}_{2} {r}^{2} + {a}_{3} {r}^{3} … {a}_{n} {r}^{n}$$

has been utilized in defining the rotationally symmetric aspheric surfaces of this invention, where z equals the surface sag along the lens axis, c equals the curvature (i.e., reciprocal of the radius), r is the radial coordinate in lens units, k equals the conic constant, and aₙ(where n=1, 2, . . . ) is the coefficient value of any selected conic deformation terms.

Referring again the FIG. 2a, it may be noted that the diameter of contact element 12 exceeds that of interface 18 thus allowing element 12 to be advantageously shaped to function as an eyelid flange. An eyelid flange facilitates a positive interface with the tear or fluid layer of the eye when the patient tends to blink or squeeze the eyelids closed during the diagnostic or treatment procedure, and the use of a flange is known to those skilled in the art. The contact elements of subsequent figures and embodiments likewise may incorporate diameters or recesses that provide a lid flange function and are shown with various flange designs that may be used.

As previously mentioned, in the lens of the present disclosure light beams proceeding through the lens from the examined eye to the inverted real image are each reflected in an ordered sequence of reflections with the first reflection occurring from a concave anterior reflecting surface a posterior direction and with the second reflection occurring from a convex posterior reflecting surface as a negative reflection in an anterior direction.

FIG. 2b shows an enlargement of reflecting element 14 and the pathway of the central ray of light beam 2 shown in FIG. 2a, proceeding through the lens from interface 18 to refracting surface 26, clearly illustrating how the reflections of light beams conform to the first stated combination of reflections comprising a positive reflection from the first reflecting surface and negative reflection from the second reflecting surface as described. Line P is perpendicular to lens axis LA and extends from intersection point LAP of line P and lens axis LA. Reflected Ray 2b proceeds from anterior reflecting surface 20 further from lens axis LA than preceding incident ray 2a as demonstrated by each ray's respective intersection point 2aP and 2bP with line P and specifically as demonstrated by the lesser distance from 2aP to LAP compared to the greater distance from 2bP to LAP. Line P1 is perpendicular to lens axis LA and extends from intersection point LAP1 of line P1 and lens axis LA. Reflected light ray 2c proceeds from posterior reflecting surface 22 closer to lens axis LA than preceding incident ray 2b as demonstrated by each ray's respective intersection point 2cP and 2bP1 with line P1 and specifically as demonstrated by the greater distance from 2bP1 to LAP1 compared to the lesser distance from 2cP to LAP1. Light beams emanating from the area of the iridocorneal angle and peripheral iris and contributing to the formation of an inverted real image each reflect in this ordered sequence of reflections in the present as well as in subsequent embodiments and examples following the first stated combination of reflection pathways. Any perpendicular line P or P1 extending from the lens axis that intersects pairs of incident and reflected rays following the above stated reflection pathways will demonstrate this property.

In the lens of the present disclosure, it is preferred that the central ray of a light beam emanating from the iridocorneal angle and following the above stated reflection pathways follows a pathway from its point of reflection from the posterior reflecting surface to the refracting surface entirely within one part of the lens. This part may be defined as a portion of the lens, containing the reflected central rays of the light beam, that is on one side of a plane that contains the lens axis and which lies orthogonal to a perpendicular line that intersects the reflected central rays. Furthermore, it is preferred that the angle formed between the central ray reflecting from the posterior reflecting surface and the lens axis be less than 15°, and more preferably be less than 8°.

Thus, referring again to FIG. 2b the pathway of central ray 2c, from its point of reflection from surface 22 to refracting surface 26, remains within part F of lens 10, defined as the portion of the lens containing reflected rays 2b and 2c on one side of plane PL, which lies orthogonal to perpendicular lines P and P1 and which contains the lens axis LA. Accordingly, central ray 2c does not intersect plane PL and does not intersect lens axis LA within lens 10, and as shown, forms an angle with lens axis LA of 6°.

In the lens of the present disclosure it is further preferred that the central ray of a light beam emanating from the iridocorneal angle and following the above first stated reflection pathways have a combined value of the angles of reflection from both the anterior and posterior reflecting surfaces less than 24.5° and preferably below 18.5°. In FIG. 2b, the combined value of the angles of reflection of central rays 2b and 2c is 17.3°.

The above stated central light ray angles and pathways of the lens of the present disclosure, described with respect to lens 10 of FIG. 2b, provide the benefit of minimizing image aberrations associated with light beams emanating from the area of the iridocorneal angle that contribute to the formation of an inverted real image and which exit through the refracting surface of the lens and span a diameter extent of at least 30 mm centered about the lens axis at a distance 100 mm anterior of the virtual image associated with the inverted real image.

As previously mentioned the annular reflecting surfaces form an aperture providing a clear central viewing portion through the lens that facilitates positioning of the lens on an examined eye and allows direct viewing and treatment of other structures of the eye. The light transmitting pathway provided through the 16 mm and 6.3 mm clear apertures of annular mirrored sections 22 and 20 respectively of lens 10 allows the practitioner to see the patient's eye through the lens as he or she looks through the biomicroscope while preparing to apply of the lens, thus the practitioner can discern the proximity of the lens to the patient's cornea with some confidence. Once the lens is applied to the patient's cornea the practitioner may direct the biomicroscope's focus to the central area of the lens in order to view structures of the eye through only the central refracting portion as defined by the clear apertures within the annular mirrored sections. The lens has a focal length in air of −64.6 mm, and once on the eye may provide a direct view as a virtual image of various structures of the eye.

Referring to FIG. 3, there is shown a ray tracing and schematic cross-sectional view of the gonioscopy lens of the first embodiment depicted in prior FIGS. 1 and 2a directed to visualization of the paracentral iris and laser iridotomy procedures through the central non-mirrored portion of the lens. A first non-reflective portion, positioned proximate to the posterior reflecting surface, and a second non-reflective portion, positioned proximate to the anterior reflecting surface, provide a transparent path through the lens that allows direct viewing by the practitioner. In one example, the first non-reflective portion may comprise a section of the curvature defining the convex posterior reflector that is inward (i.e., toward the lens axis) of the annular shape of the posterior reflector. In another example, the first non-reflective portion may comprise any other shape and be positioned along the lens axis proximate to the convex posterior reflector. Likewise, the second non-reflective portion may comprise the section of the curvature defining the concave anterior reflector that is inward of the annular shape of the anterior reflector. In another example, the second non-reflective portion may comprise any other shape and be positioned along the lens axis proximate to the concave anterior reflector. It will be understood that a non-reflective portion is proximate to a reflector even when it is spaced away from and not in contact with the reflector. A non-reflective portion may be displaced a distance away from a reflector, for example a several millimeters along the lens axis, and still be positioned proximate to the reflector. To focus on the iris structures through the central lens aperture the biomicroscope may be moved in a forward direction relative to its position when focused on the inverted image of the iridocorneal angle and peripheral iris with the mirror system. Used in this manner lens 10 reduces the power of the eye and provides a 1.76 image magnification and 0.568 laser spot reduction. The virtual image of the iris thus produced is located approximately at the plane of the posterior surface of the crystalline lens.

Referring to FIG. 3, light rays 36a and 36b emanating from paracentral iris locations 38a and 38b respectively of anterior chamber 4 of eye 6 pass through the cornea 8 and tear layer of the eye and enter lens element 12 of lens 10 through corneal contacting surface 16 and continue through the 6.3 mm aperture of interface 18 into anterior lens element 14 and to the 16 mm aperture of refracting surface 26 where they exit the lens and proceed to the biomicroscope. The biomicroscope is focused at virtual image plane 39 to provide an upright and correctly oriented direct view of the observed structures of the iris.

Lens 10 may be similarly used in capsulotomy applications by repositioning the biomicroscope to focus on the posterior capsule. The virtual image of the posterior capsule thus will be located approximately seven millimeters posterior of the physical structures observed.

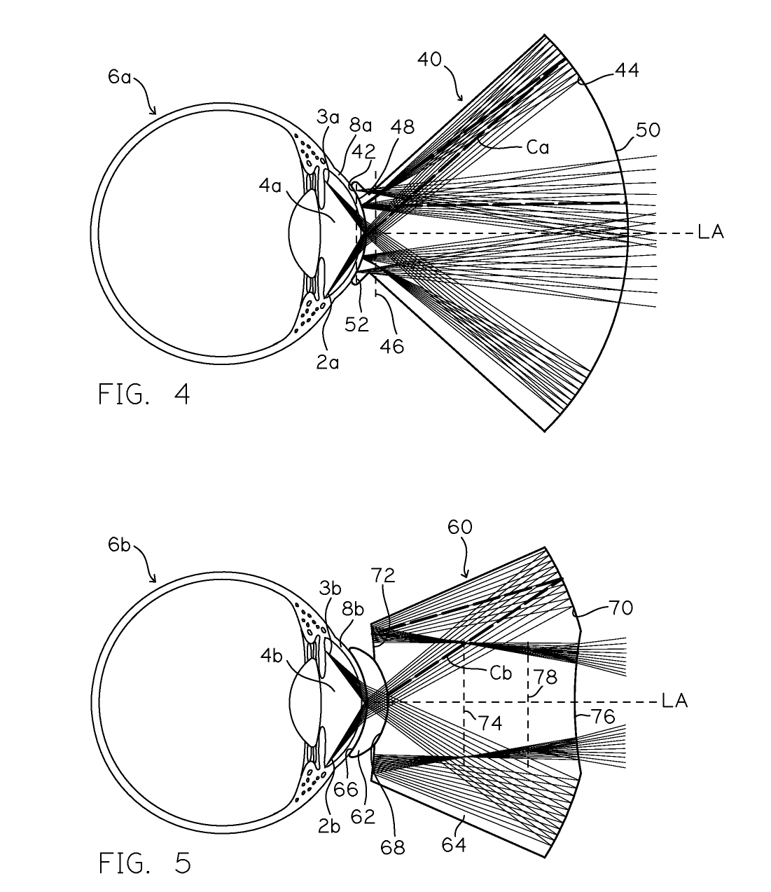

Referring to FIG. 4, there is shown a ray tracing and schematic cross-sectional view of an exemplary single element gonioscopy lens 40 according to a second embodiment of the invention. In this embodiment the anterior reflecting surface comprises an aspheric curvature and the posterior reflecting surface comprises a spherical surface. The lens is made of optical quality polycarbonate having an index of refraction of approximately Nd=1.585 and an Abbe number of approximately Vd=29.9.

Referring to FIG. 4, light beams 2a and 3a emanating from the prior stated iridocorneal and peripheral iris locations of anterior chamber 4a of eye 6a pass through the cornea 8a and tear layer of the eye and enter lens 40 through corneal contacting surface 42 and continue to concave annular reflecting surface 44 from which each light beam is first reflected as a positive reflection in a posterior direction. The convergent light beams proceed in their respective directions and focus at dotted line 46, which represents the plane of the inverted real image. The light beams continue from inverted image 46 to convex annular mirror surface 48 from which each light beam is next reflected as a negative reflection in an anterior direction towards refracting surface 50 where they are refracted and exit the lens. Virtual image 52, which is the apparent location of real image 46, is located 26 mm posterior of surface 50. With respect to light beam 2a, the value for the combined angles of reflection of central ray Ca from reflecting surfaces 44 and 48 is approximately 20.6°, the angle formed between central ray Ca and lens axis LA after its reflection from surface 48 is approximately 1.8°, and the span of beam 2a 100 mm anterior of virtual image 52 exceeds a 30 mm diameter centered about lens axis LA.

Contacting surface 42 comprises a concave surface adapted for placement on the patient's cornea, and may have a spherical or aspherical curvature. In the exemplary lens of this embodiment surface 42 has a radius of 8.0 and is spherical. Reflecting surface 44 has an aspheric concave curvature with an apical radius of 26.3 mm and in combination with refracting surface 50 comprises a continuous aspheric curvature as the anterior surface of lens 40. Reflecting surface 44 comprises an internally reflecting mirror-coated annular section having a 21 mm inner diameter that surrounds refracting surface area 50. Annular reflecting surface 48 has a convex spherical curvature with a radius of 8.0 mm and in combination with contacting surface 42 comprises a continuous spherical curvature as the posterior surface of the lens. Reflecting surface 48 also comprises a mirror-coated annular section having a 4 mm inner diameter that surrounds the refracting area of contacting surface 42. The reflective sections may be mirrored and protectively overcoated by means previously mentioned. Annular mirrored section 48 together with transparent central contacting portion 42 may have a continuous polymeric layer applied by spin coating or cast as a thin replicated surface layer.

The exemplary lens as shown and described with reference to FIG. 4, comprising a first anterior plus powered aspheric reflector paired with a second posterior minus powered spherical reflector, each which respectively produce the stated posterior and positive and anterior and negative reflections, provides a single element optical system for a diagnostic and therapeutic gonioscopy lens with excellent imaging qualities that exhibits simplicity of design and ease of manufacture due to its polymeric material composition, single element design and single aspheric surface.

Referring to FIG. 5, there is shown a ray tracing and schematic cross-sectional view of an exemplary doublet gonioscopy lens according to a third embodiment of the invention, wherein lens 60 comprises an optically coupled lens including posterior contacting element 62 and anterior element 64. In this embodiment both the posterior and anterior surfaces of lens element 64 comprise lenticulated surfaces, and all the refracting and reflecting surfaces are spherical. Both posterior element 62 and anterior element 64 are made of S-LAH59 glass (available from Ohara Corp.) with an index of refraction of approximately Nd=1.816 and an Abbe number of approximately Vd=46.6. As a cemented doublet, the two elements 62 and 64 may be adhered together at their interface using optical adhesive OP-4-20658 manufactured by Dymax Corporation.

Referring to FIG. 5, light beams 2b and 3b emanating from the stated iridocorneal and peripheral iris locations of anterior chamber 4b of eye 6b pass through the cornea 8b and tear layer of the eye and enter posterior contacting element 62 of lens 60 through corneal contacting surface 66 and continue through interface 68, comprised of the anterior and posterior surfaces of lens elements 62 and 64 respectively, optically coupled with an interface material. The light beams continue to concave annular reflecting surface 70 from which each light beam is first reflected as a positive reflection in a posterior direction. The convergent light beams proceed in their respective directions to convex annular reflecting surface 72 from which each light beam is next reflected as a negative reflection in an anterior direction. Proceeding from reflecting surface 72 the light beams focus at real inverted image 74 and continue in their respective directions towards surface 76 where they are refracted and exit the lens. Virtual image 78, which is the apparent location of real image 74, is located 4.65 mm posterior of surface 76. With respect to light beam 2b, the value for the combined angles of reflection of central ray Cb from reflecting surfaces 70 and 72 is approximately 20.15°, the angle formed between central ray Cb and lens axis LA after its reflection from surface 72 is approximately 5.8°, and the span of beam 2b 100 mm anterior of virtual image 78 exceeds a 30 mm diameter centered about lens axis LA.

Contacting surface 66 comprises a concave surface adapted for placement on the patient's cornea, and may have a spherical or aspherical curvature. In the exemplary lens of this embodiment surface 66 has a radius of 8.0 mm and is spherical. Optical interface 68 is the interface of the central refracting portions of the anterior and posterior surfaces respectively of lens elements 62 and 64. The curvature of interface 68 with respect to lens element 64 is spherical and concave with a radius of 7.0 mm. Reflecting surface 70 has a concave spherical curvature with an apical radius of 28.1 mm and together with refracting surface 76 comprises a lenticulated surface as the anterior surface of lens element 64. Refracting surface 76 has a concave spherical curvature with an apical radius of 30 mm. Reflecting surface 70 comprises an internally reflecting mirror-coated annular section having a 13.5 mm inner diameter that surrounds refracting surface area 76. Annular reflecting surface 72 has a convex spherical curvature with an apical radius of 66.5 mm and together with refracting surface area 68 comprises a lenticulated surface as the posterior surface of lens element 64. Reflecting surface 72 also comprises an internally reflecting mirror-coated annular section having an 8.15 mm inner diameter that surrounds refracting surface area 68. The reflective sections may be mirrored by means previously mentioned.

The exemplary lens as shown and described with reference to FIG. 5, comprising a first anterior plus powered spherical reflector paired with a second posterior minus powered spherical reflector, each which respectively produce the stated posterior and positive and anterior and negative reflections, provides a two-element optical system for a diagnostic and therapeutic gonioscopy lens with excellent imaging qualities utilizing lenticulated designs for both anterior lens surface and the surface incorporating the posterior reflector and the refracting portion it surrounds, all surfaces having spherical curvatures.

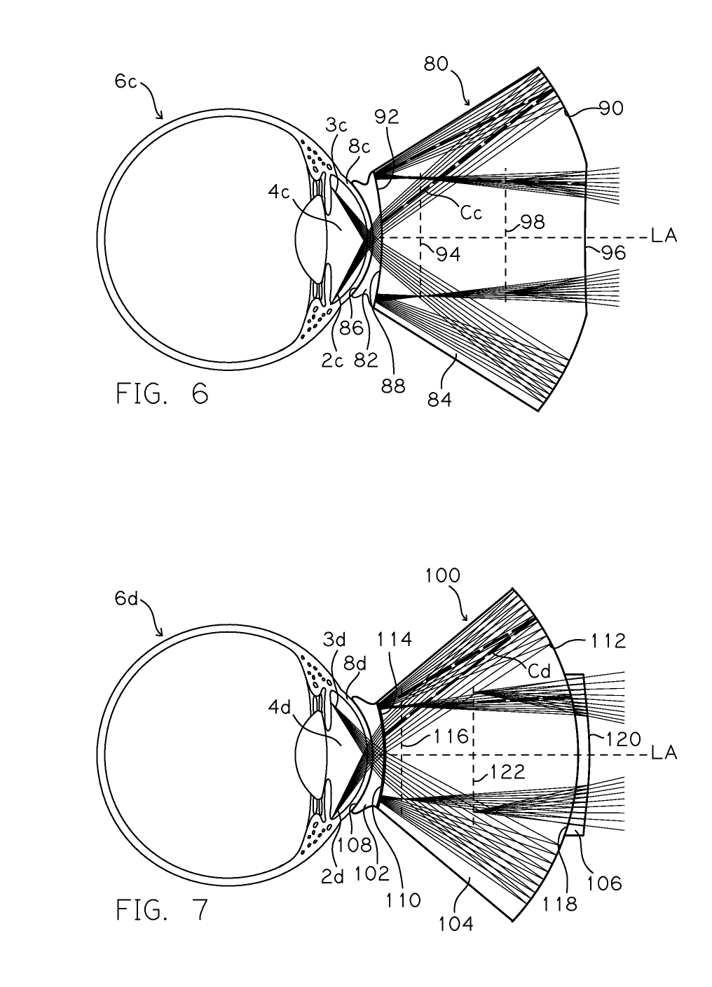

Referring to FIG. 6, there is shown a ray tracing and schematic cross-sectional view of an exemplary doublet gonioscopy lens according to a fourth embodiment of the invention, wherein lens 80 comprises an optically coupled lens including posterior contacting element 82 and anterior element 84. In this embodiment the anterior surface of lens element 84 comprises a lenticulated surface and all refracting and reflecting surfaces are spherical. Posterior contacting element 82 is made of optical quality polymethylmethacrylate and anterior element 84 is made of S-LAH58 optical glass. As a cemented doublet, the two elements 82 and 84 may be adhered together at their interface using 3-20261 optical cement.

Referring to FIG. 6, light beams 2c and 3c emanating from the stated iridocorneal and peripheral iris locations of anterior chamber 4c of eye 6c pass through the cornea 8c and tear layer of the eye and enter posterior contacting element 82 of lens 80 through corneal contacting surface 86 and continue through interface 88, comprised of the anterior and posterior surfaces of lens elements 82 and 84 respectively, optically coupled with an interface material. The light beams continue to concave annular reflecting surface 90 from which each light beam is first reflected as a positive reflection in a posterior direction. The convergent light beams proceed in their respective directions to convex annular reflecting surface 92 from which each light beam is next reflected as a negative reflection in an anterior direction. Proceeding from reflecting surface 92 the light beams focus at real inverted image 94 and continue in their respective directions towards surface 96 where they are refracted and exit the lens. Virtual image 98, which is the apparent location of real image 94, is located 8.15 mm posterior of surface 96. With respect to light beam 2c, the value for the combined angles of reflection of central ray Cc from reflecting surfaces 90 and 92 is approximately 20.8°, the angle formed between central ray Cc and lens axis LA after its reflection from surface 92 is approximately 2.4°, and the span of beam 2c 100 mm anterior of virtual image 98 exceeds a 30 mm diameter centered about lens axis LA.

Contacting surface 86 comprises a concave surface adapted for placement on the patient's cornea, and may have a spherical or aspherical curvature. In the exemplary lens of this embodiment surface 86 has a radius of 8.0 mm and is spherical. Optical interface 88 is the interface of the central refracting portions of the anterior and posterior surfaces respectively of lens elements 82 and 84. The curvature of interface 88 with respect to lens element 84 is spherical and concave with a radius of 28.5 mm. Reflecting surface 90 has a concave spherical curvature with an apical radius of 27 mm and together with refracting surface 96 comprises a lenticulated surface as the anterior surface of lens element 84. Refracting surface 96 has a concave spherical curvature with an apical radius of 158.4 mm. Reflecting surface 90 comprises an internally reflecting mirror-coated annular section having a 14.5 mm inner diameter that surrounds refracting surface area 96. Annular reflecting surface 92 has a convex spherical curvature with an apical radius of 28.5 mm and together with refracting surface area 88 comprises a continuous surface as the posterior surface of lens element 84. Reflecting surface 92 also comprises an internally reflecting mirror-coated annular section having a 10 mm inner diameter that surrounds refracting surface area 88. The reflective sections may be mirrored by means previously mentioned.

The exemplary lens as shown and described with reference to FIG. 6, comprising a first anterior plus powered spherical reflector paired with a second posterior minus powered spherical reflector, each which respectively produce the stated posterior and positive and anterior and negative reflections, provides a two-element optical system for a diagnostic and therapeutic gonioscopy lens with excellent imaging qualities utilizing a lenticulated design for the anterior lens surface and a surface of continuous curvature for the surface incorporating the posterior reflector and the refracting portion it surrounds, all surfaces having spherical curvatures.

Referring to FIG. 7 there is shown a ray tracing and schematic cross-sectional view of an exemplary triplet gonioscopy lens according to a fifth embodiment of the invention, wherein lens 100 comprises an optically coupled lens including posterior element 102, middle element 104 and anterior element 106. In this embodiment the reflecting and refracting surfaces comprising the anterior surface of middle lens element 104 together comprise a spherical surface, the posterior reflecting surface comprises the anterior surface of lens element 102 and is aspheric, the posterior surface of middle element 104 is spherical, and the anterior surface of lens element 106 is aspheric. Posterior element 102 is made of optical quality polymethylmethacrylate, middle element 104 is made of S-LAH58 optical glass and anterior element 106 is made of polymethylmethacrylate. As a cemented triplet, elements 102 and 104 may be adhered together at their interface with OP-4-20658 optical adhesive and elements 104 and 106 may be adhered together at their interface with 3-20261 optical adhesive.

Referring to FIG. 7, light beams 2d and 3d emanating from the stated iridocorneal and peripheral iris locations of anterior chamber 4d of eye 6d pass through the cornea 8d and tear layer of the eye and enter posterior contacting element 102 of lens 100 through corneal contacting surface 108 and continue through interface 110, comprised of the anterior and posterior surfaces of lens elements 102 and 104 respectively, optically coupled with an interface material. The light beams proceed through middle lens element 104 to concave annular reflecting surface 112 from which each light beam is first reflected as a positive reflection in a posterior direction. The convergent light beams proceed in their respective directions to convex annular reflecting surface 114 from which each light beam is next reflected as a negative reflection in an anterior direction. Proceeding from reflecting surface 114 the light beams focus at real inverted image 116 and continue through interface 118, comprised of the anterior and posterior surfaces of lens elements 104 and 106 respectively, optically coupled with an interface material. The light beams proceed through anterior lens element 106 in their respective directions towards surface 120 where they are refracted and exit the lens. Virtual image 122, which is the apparent location of real image 116, is located 11.2 mm posterior of surface 120. With respect to light beam 2d, the value for the combined angles of reflection of central ray Cd from reflecting surfaces 112 and 114 is approximately 17.7°, the angle formed between central ray Cd and lens axis LA after its reflection from surface 114 is approximately 3.1°, and the span of beam 2d 100 mm anterior of virtual image 122 exceeds a 30 mm diameter centered about lens axis LA.

Contacting surface 108 comprises a concave surface adapted for placement on the patient's cornea, and may have a spherical or aspherical curvature. In the exemplary lens of this embodiment surface 108 has a radius of 8.0 mm and is spherical. Optical interface 110 comprises the generally matched but slightly differently shaped anterior and posterior surfaces respectively of lens elements 102 and 104. Both the anterior surface of lens element 102 and the posterior surface of lens element 104 comprise surfaces of continuous curvature, with the slight curvature difference of each resulting in a thickness deviation over the extent of the interface medium of less than 0.07 mm. The posterior surface of lens element 104 is refractive over its extent and has a spherical curve with a radius of 16.15 mm. Reflecting surface 112 has a spherical concave curvature with a radius of 19.89 mm and together with the anterior refracting portion of lens element 104 of optical interface 118 comprises a continuous spherical surface as the anterior surface of lens element 104. Reflecting surface 112 comprises an internally reflecting mirror-coated annular section having a 13.8 mm inner diameter that surrounds refracting surface area 118. Annular reflecting surface 114 has a convex aspheric curvature with an apical radius of 20.44 mm and together with the anterior refracting portion of lens element 102 of optical interface 110 comprises a continuous aspheric curvature as the anterior surface of lens element 102. Annular reflecting surface 114 comprises an externally reflecting mirror-coated annular section having a 6 mm inner diameter. Reflecting surface 114 reflects incident light refracted through the posterior surface of lens element 104 and optical interface 110 in an anterior direction, through the interface medium of interface 110 and the posterior surface of lens element 104. The reflective sections may be mirrored by means previously mentioned. The posterior surface of lens element 106 is concave and spherical with a radius of 19.89 mm and anterior surface 120 is centrally convex and aspheric with an apical radius of 32.92 mm.

The exemplary lens as described, comprising an anterior plus powered spherical reflector paired with a posterior minus powered spherical reflector, each which respectively produce the stated posterior and positive and anterior and negative reflections, provides a three element optical system for a diagnostic and therapeutic gonioscopy lens with excellent imaging qualities that utilizes a middle glass element easily made with spherical anterior and posterior surfaces and posterior and anterior acrylic elements utilizing polynomial aspheric surfaces providing optimum correction of aberrations that are likewise easily manufactured.

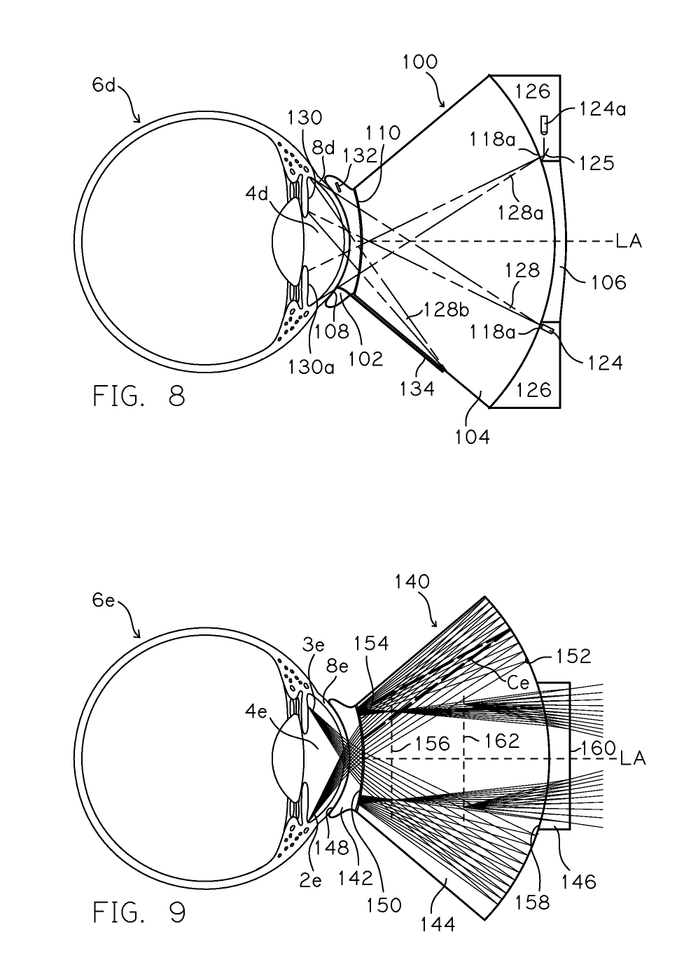

Referring to FIG. 8 there is shown a schematic cross-sectional view of the gonioscopy lens of the fifth embodiment depicted in prior FIG. 7, directed to various illumination systems that may be used as an alternative to or in addition to illumination provided by the slit lamp biomicroscope. As previously mentioned light-guiding optical fibers positioned in relationship to the contacting element or light-emitting diodes directing illumination directly through the lens to the structures of the anterior chamber may be utilized. A series or array of OLED or LED lamps may be positioned in a ring formation at the anterior end of the lens, directing emitted light through an annular area between the inner aspect of the anterior reflecting surface and the refracting portion through which light beams proceed through and exit the lens. The illuminating light may follow similar but oppositely directed pathways to light beams emanating from the anterior chamber and proceeding to the anterior reflecting surface. Alternatively, the illumination lamps may be positioned at a mid-way point between the posterior and anterior ends of the lens, and direct their illumination through the tapered side of the lens. As a further alternative, extremely small and thin chip LEDs (PICOLED™, available from ROHM Co., Ltd., and measuring 1 mm×0.6 mm×0.2 mm) arranged in a ring pattern may be at least partially embedded in a portion of the lens adjacent the contacting surface, directing illumination through the contacting surface and cornea directly to the anterior chamber structures. The illumination source(s) for the optical fibers may be located at a mid-way point between the posterior and anterior ends of the lens, at the anterior end of the lens or may be remotely located. The optical fibers may be positioned adjacent the frustoconically shaped lens and terminate at or within the contacting element. As an alternative to the use of multiple fibers, a frustoconically shaped light guide fitted as a jacket around the tapering lens may provide internally reflected light through its length from its anterior larger end to the smaller posterior end where it contacts or otherwise joins the contacting element. The alternative illumination systems may advantageously provide continuous illumination of the illuminated structures even with movement of the biomicroscope during image scanning, and further may limit the amount of light passing through the pupil that illuminates the retina, thereby reducing patient discomfort and glaring slit lamp light source reflections from optical surfaces that interfere with diagnostic and treatment procedures and which are disturbing to the practitioner. Some of the alternative illumination systems may be designed to affix to or be detachably removable from the opthalmoscopic contact lens and may be utilized in conjunction with the lens of the present embodiment as well as those of subsequent embodiments. In the following FIG. 8, various illumination systems as above described are shown without electric wire connections, power supply and a fiber optic light source, as it is understood that such auxiliary systems may be incorporated in manners typically employed.

Referring to FIG. 8, LED 124 is positioned adjacent the anterior surface of lens element 104 and may be mounted within carrier 126 which encircles anterior lens element 106. Carrier 126 may be made of Delrin® acetyl plastic or other suitable material. The LED beam may be directed at an angle of approximately 30° with respect to lens axis LA and have an output beam angle of approximately 7° in order to limit the area of illumination to that of the iris width 130. Emitted LED illumination beam 128 enters lens element 104 through refracting surface area 118a, passes through interface 110, contacting element 102, cornea 8d and anterior chamber 4d of eye 6d to illuminate iris and iridiocorneal areas 130. A circular array of LEDs providing illumination as above described and positioned in a ring pattern around lens 106 may provide illumination of the entire iris and iridiocorneal angle. Electrical power may be provided by battery (electric cell) located in a compartment such as carrier 126 or supplied by attached wire (not shown). LED 124a shows an alternative lamp orientation generally orthogonal to the lens axis in which light is directed to mirror 125 and reflected at the same 30° angle as previously described. Emitted beam 128a enters lens element 104, passes through interface 110, contacting element 102, cornea 8d and anterior chamber 4d of eye 6d to illuminate iris and iridiocorneal areas 130a. A circular array of LEDs and mirror sections may provide illumination of the entire iris and iridiocorneal angle as previously described. As mentioned, an alternative location between the posterior and anterior ends of the frustoconically shaped lens may provide a series of LEDs similarly arranged (not shown) to direct emitted light along a more highly angulated pathway represented by 128b, through contacting element 102, cornea 8d and to the anterior chamber structures 130. A further arrangement mentioned above comprises a series of extremely small LEDs, represented by LED 132, positioned in a ring formation within contacting element 102 or a portion of the lens adjacent the cornea, directing emitted light (not shown) through contacting surface 108 and cornea 8d to illuminate the structures of the anterior chamber 130. A further alternative method utilizing optical fibers positioned around and adjacent frustoconically shaped lens 100 is represented by optical fiber 134, which is shown entering contacting element 102 to provide illumination of the anterior chamber structures 130a through cornea 8d. Optical fiber 134 may terminate at or within contacting element 102.

Referring to FIG. 9, there is shown a ray tracing and schematic cross-sectional view of an exemplary triplet gonioscopy lens according to a sixth embodiment of the invention, wherein lens 140 comprises an optically coupled lens including posterior contacting element 142, middle element 144 and anterior element 146. In this embodiment the reflecting and refracting surfaces comprising the anterior surface of middle lens element 144 each comprise different aspheric curvatures that join tangentially and without discontinuity, the posterior reflecting surface comprises the anterior surface of lens element 142 and is aspheric, the posterior surface of middle element 144 is spherical, and the anterior surface of lens element 146 is plano. Posterior element 142 is made of optical quality polymethylmethacrylate, middle element 144 is made of S-LAH58 optical glass and anterior element 146 is made of S-FPL51Y (available from Ohara Corp.) having an index of refraction of approximately Nd=1.497 and an Abbe number of approximately Vd=81.14. As a cemented triplet, elements 142 and 144 may be adhered together at their interface with OP-4-20658 optical adhesive and elements 144 and 146 may be adhered together at their interface with 3-20261 optical adhesive.

Referring to FIG. 9, light beams 2e and 3e emanating from the stated iridocorneal and peripheral iris locations of anterior chamber 4e of eye 6e pass through the cornea 8e and tear layer of the eye and enter posterior contacting element 142 of lens 140 through corneal contacting surface 148 and continue through interface 150, comprised of the anterior and posterior surfaces of lens elements 142 and 144 respectively, optically coupled with an interface material. The light beams proceed through middle lens element 144 to concave annular reflecting surface 152 from which each light beam is first reflected as a positive reflection in a posterior direction. The convergent light beams proceed in their respective directions to convex annular reflecting surface 154 from which each light beam is next reflected as a negative reflection in an anterior direction. Proceeding from reflecting surface 154 the light beams focus at real inverted image 156 and continue through interface 158, comprised of the anterior and posterior surfaces of lens elements 144 and 146 respectively, optically coupled with an interface material. The light beams proceed through anterior lens element 146 in their respective directions towards surface 160 where they are refracted and exit the lens. Virtual image 162, which is the apparent location of real image 156, is located 10.15 mm posterior of surface 160. With respect to light beam 2e, the value for the combined angles of reflection of central ray Ce from reflecting surfaces 152 and 154 is approximately 18.3°, the angle formed between central ray Ce and lens axis LA after its reflection from surface 154 is approximately 1.1°, and the span of beam 2e 100 mm anterior of virtual image 162 exceeds a 30 mm diameter centered about lens axis LA.

Contacting surface 148 comprises a concave surface adapted for placement on the patient's cornea, and may have a spherical or aspherical curvature. In the exemplary lens of this embodiment surface 148 has a radius of 8.0 mm and is spherical. Optical interface 150 comprises the generally matched but slightly differently shaped anterior and posterior surfaces respectively of lens elements 142 and 144. Both the anterior surface of lens element 142 and the posterior surface of lens element 144 comprise surfaces of continuous curvature, with the slight curvature difference of each resulting in a thickness deviation over the extent of the interface medium of less than 0.07 mm. The posterior surface of lens element 144 is refractive over its extent and has a spherical curvature with a radius of 17.81 mm. Anterior reflecting surface 152 has an aspheric concave curvature with an apical radius of 21.88 mm and together with the anterior refracting portion of lens element 144 of optical interface 158 forms a continuous curvature comprising two aspheric surfaces that join tangentially as the anterior surface of lens element 144. Reflecting surface 152 comprises an internally reflecting mirror-coated annular section having a 13.65 mm inner diameter that surrounds refracting surface area 158. Annular reflecting surface 154 has a convex aspheric curvature of increasing curvature with an apical radius of 14.32 mm and together with the anterior refracting portion of lens element 142 of optical interface 150 comprises a continuous aspheric curvature as the anterior surface of lens element 142. Reflecting surface 154 comprises an externally reflecting mirror-coated annular section having a 6.8 mm inner diameter. Reflecting surface 154 reflects incident light refracted through the posterior surface of lens element 144 and optical interface 150 in an anterior direction, through the interface medium of interface 150 and the posterior surface of lens element 144. The reflective sections may be mirrored by means previously mentioned. Optical interface 158 comprises the generally matched but slightly differently shaped anterior and posterior surfaces respectively of lens elements 144 and 146. The anterior refracting portion of lens element 144 of optical interface 158 has a convex aspheric curvature with an apical radius of 21.7 mm. The posterior surface of lens element 146 is concave and spherical with a radius of 21.5 mm. The slight curvature difference of each results in a thickness deviation over the extent of the interface medium of interface 158 of less than 0.02 mm. Anterior surface 160 of lens element 146 is plano. Anterior lens element 146 is shown having a diameter less than that of lens element 144 at its anterior end. Lens element 146 may alternatively be as large as lens element 144 in which case mirror surface 152 may be protected within larger interface 158. Prior to interfacing lens elements 144 and 146, mirror coating 152 may be overcoated to eliminate reflection externally.

The exemplary lens as described, comprising an anterior plus powered aspheric reflector paired with a posterior minus powered aspheric reflector, each which respectively produce the stated posterior and positive and anterior and negative reflections, provides a three element optical system for a diagnostic and therapeutic gonioscopy lens with excellent imaging qualities that utilizes a continuous bi-aspheric anterior surface and spherical concave posterior surface for the middle element and a spherical plano-concave anterior element.

As previously mentioned the lens may be designed with a clear central viewing portion through the lens that facilitates positioning of the lens on an examined eye and allows direct viewing and treatment of other structures of the eye. The light transmitting pathway provided through the 13.65 mm and 6.8 mm clear apertures of annular mirrored sections 152 and 154 respectively of lens 140 allows the practitioner to see the patient's eye through the lens as he or she looks through the biomicroscope while preparing to apply of the lens, thus the practitioner can discern the proximity of the lens to the patient's cornea with some confidence. Once the lens is applied to the patient's cornea the practitioner may direct the biomicroscope's focus to the central area of the lens in order to view structures of the eye through only the central refracting portion as defined by the clear apertures within the annular mirrored sections. The lens has a focal length in air of −18.9 mm, and once on the eye may provide a direct view as a virtual image of various structures of the eye.

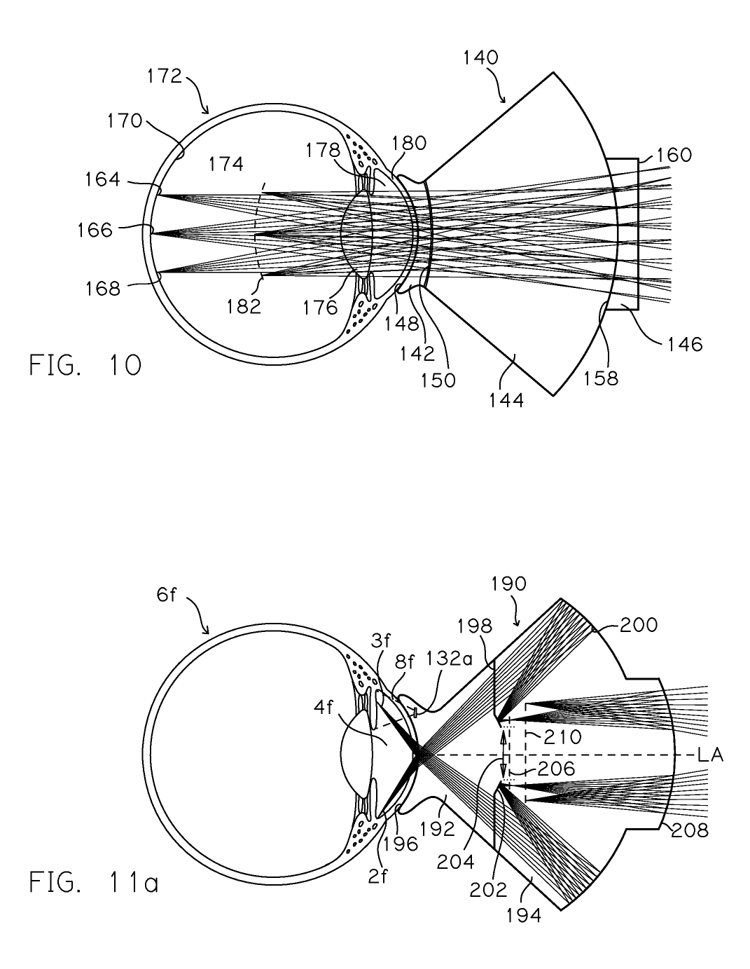

Referring to FIG. 10, there is shown a ray tracing and schematic cross-sectional view of the gonioscopy lens of the sixth embodiment depicted in prior FIG. 9 directed to diagnosis of the macula and central retina and focal laser procedures through the central non-mirrored portion of the lens. To focus on fundus structures through the central lens aperture the biomicroscope may be moved in a forward direction relative to its position when focused on the inverted image of the iridocorneal angle and peripheral iris with the mirror system. Used in this manner lens 140 reduces the power of the eye and provides a 1.09 image magnification and 0.913 laser spot reduction. The virtual image of the fundus thus produced is located anterior of the observed structures.

Referring to FIG. 10, light rays 164, 166 and 168 emanating from points on the retina 170 of eye 172 pass through the vitreous humor 174, crystalline lens 176, anterior chamber 178, cornea 180 and tear layer of the eye and enter lens element 142 through contacting surface 148. The light rays pass through the 6.8 mm aperture of optical interface 150 into lens element 144 and continue through the 13.6 mm aperture of optical interface 158 into lens element 146 and exit the lens through anterior surface 160 from which they proceed towards the biomicroscope. The biomicroscope is focused at virtual image 182 to provide an upright and correctly oriented direct view of the fundus.

As previously mentioned a second group of embodiments provides a lens construction in which a light beam proceeding through the lens from the examined eye to the inverted real image is reflected in an ordered sequence of reflections first as a negative reflection in a posterior direction from the anterior concave reflecting surface and next as a negative reflection in an anterior direction from a convex posterior reflecting surface with each reflecting surface being formed as an annulus. A first non-reflective portion can be positioned along the lens axis and proximate to the posterior reflecting surface, and a second non-reflective portion can be positioned along the lens axis and proximate to the anterior reflecting surface. Such an arrangement can provide for the transmission of light directly through the lens, and additionally can prevent glaring slit lamp light source reflections from optical surfaces from interfering with diagnostic and treatment procedures, which can be disturbing to the practitioner.

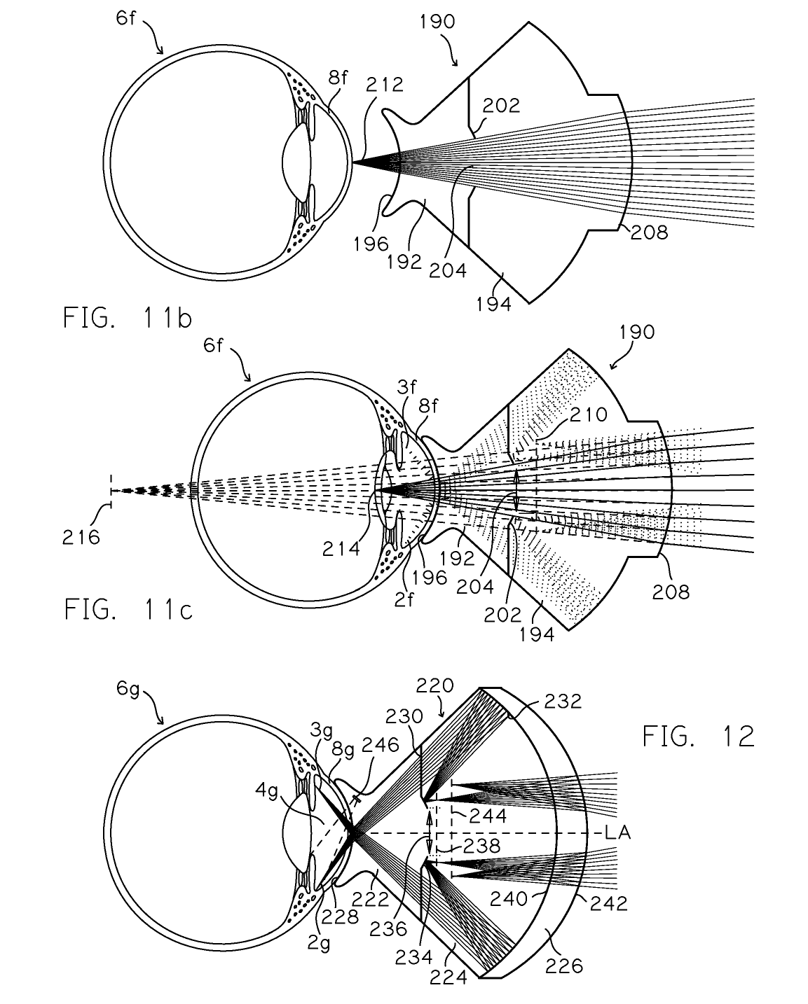

Referring to FIG. 11a, there is shown a ray tracing and schematic cross-sectional view of an exemplary doublet gonioscopy lens according to a seventh embodiment of the invention, wherein lens 190 comprises an optically coupled lens including posterior contacting element 192 and anterior element 194. In this embodiment the anterior surface of lens element 194 comprises a lenticulated surface, the refracting and reflecting surfaces are aspheric and the posterior reflecting surface is formed as an annulus in which the non-reflective central area or portion provides transmission of light directly through the lens. Both posterior element 192 and anterior element 194 are made of optical quality polycarbonate. As a cemented doublet, the two elements 192 and 194 may be adhered together at their interface using optical adhesive OP-4-20658.

Referring to FIG. 11a, light beams 2f and 3f emanating from the stated iridocorneal and peripheral iris locations of anterior chamber 4f of eye 6f pass through the cornea 8f and tear layer of the eye and enter posterior contacting element 192 of lens 190 through corneal contacting surface 196 and continue through interface 198, comprised of the anterior and posterior surfaces of lens elements 192 and 194 respectively, optically coupled with an interface material. The light beams continue to concave annular reflecting surface 200 from which each light beam is first reflected as a negative reflection in a posterior direction. The convergent light beams proceed in their respective directions to convex annular reflecting surface 202 defined by inner diameter aperture 204 from which each light beam is next reflected as a negative reflection in an anterior direction. Proceeding from annular reflecting surface 202 the light beams focus at real inverted image 206 and continue in their respective directions towards surface 208 where they are refracted and exit the lens. Virtual image 210, which is the apparent location of real image 206, is located 14.1 mm posterior of surface 208 and the span of beam 2f 100 mm anterior of virtual image 208 exceeds a 30 mm diameter centered about lens axis LA.

Contacting surface 196 comprises a concave surface adapted for placement on the patient's cornea, and may have a spherical or aspherical curvature. In the exemplary lens of this embodiment surface 196 has a radius of 8.0 mm and is spherical. Optical interface 198 is the interface of the peripheral and central refracting portions of the anterior and posterior surfaces respectively of lens elements 192 and 194 and includes aperture 204. The peripheral refracting portion of interface 198 is plano. Reflecting surface 200 has a concave aspheric curvature with an apical radius of 18.1 mm and together with refracting surface 208 comprises a lenticulated surface as the anterior surface of lens element 194. Refracting surface 208 has a convex aspheric curvature with an apical radius of 17.52 mm. Reflecting surface 200 comprises an internally reflecting mirror-coated annular section having a 20 mm inner diameter that surrounds the outside diameter of anteriorly displaced refracting surface 208, which serves to unify and precisely position the left and right eye images comprising the stereoscopic view across the extent of the visualized field and provide increased magnification of the observed image. The virtual image thus formed is magnified over the inverted real image by a factor of approximately 1.49. Annular reflecting surface 202 has a convex aspheric curvature with an apical radius of 5.54 mm and together with aperture 204 and plano refracting surface area 198 comprises a lenticulated surface as the posterior surface of lens element 194. Reflecting surface 202 also comprises an internally reflecting mirror-coated annular section having a 7.1 mm outer diameter and a 5.0 mm inner diameter that surrounds refracting aperture 204. Aperture 204 is the clear central area within posterior annular reflector 202 and has a diameter of 5.0 mm. The reflective sections may be mirrored by means previously mentioned. As an alternative to polycarbonate, polymethylmethacrylate or other polymeric or glass materials may be utilized as the material composition of the posterior and anterior lens elements. Furthermore, the scale of the lens may be modified to provide increased or decreased magnification, and the anteriorly displaced refracting surface may be displaced a greater or lesser amount, and may comprise a surface that is continuous with that of the anterior reflecting surface. Illumination of the anterior chamber may be provided by LED 132a, shown positioned adjacent contacting surface 196 as previously described.

As previously stated, the light transmitting pathway provided by an aperture through the inner diameter of the annular posterior reflecting surface allows the practitioner to see the patient's eye through the lens as he or she looks through the biomicroscope while preparing to apply of the lens, thus the practitioner can discern the proximity of the lens to the patient's cornea. FIG. 11b shows a ray tracing and schematic cross-sectional view of the gonioscopy lens of the seventh embodiment depicted in prior FIG. 11a directed to visualization of the exterior eye through the central non-mirrored portion of the lens when positioned in air and generally in alignment with a biomicroscope. For example, to focus on the cornea of the eye through the central lens aperture with the contacting surface of the lens positioned in air 5 mm from the patient's cornea, the biomicroscope may be moved in a forward direction approximately 24 mm relative to its position when it is focused without the lens directly on the surface of the cornea, thus the biomicroscope is focused at a virtual image of the eye structure. The converging power of surface 208 reduces the slit lamp working distance even though the focal length of lens 190 in air is approximately −502 mm.

Referring to FIG. 11b, light beam 212, emanating from the surface of cornea 8f of eye 6f, proceeds in an anterior direction and enters contacting element 192 of lens 190 through contacting surface 196 and continues through aperture 204 within the inner diameter of annular posterior reflector 202. Light beam 212 continues through aperture 204 into anterior lens element 194 and to refracting surface 208 where it exits the lens and proceeds approximately 46.4 mm to the biomicroscope objective lens aperture, which is par focal with the indicated light beam pathway. Thus the practitioner may easily view the exterior eye through the lens in air with the biomicroscope prior to and in preparation of a diagnostic or treatment procedure.

Referring to FIG. 11c, there is shown a ray tracing and schematic cross-sectional view of the gonioscopy lens of the seventh embodiment depicted in prior FIGS. 11a and 11b directed to examination of the posterior capsule through the central non-mirrored portion of the lens. As previously mentioned, a first non-reflective portion, positioned proximate to the posterior reflecting surface, and a second non-reflective portion, positioned proximate to the anterior reflecting surface, provide a transparent path through the lens that allows direct viewing by the practitioner. For reference, light beams 2f and 3f are shown as dotted lines following the reflected pathways to inverted real image 210. To focus on the posterior capsule through the central lens aperture the biomicroscope may be moved in a forward direction relative to its position when focused on the inverted image of the iridocorneal angle and peripheral iris with the mirror system. Used in this manner lens 190 reduces the power of the eye and provides a 2.7 image magnification of the capsular structures. The virtual image of the posterior capsule thus produced is located where the convergent dashed lines focus at virtual image plane 216. Light beam 214 emanates from the surface of the posterior capsule of eye 6f and passes through the intraocular lens and anterior chamber (not identified), and continues through cornea 8f and enters contacting element 192 of lens 190 through contacting surface 196 and continues through aperture 204 within the inner diameter of annular posterior reflector 202. Light beam 214 continues through aperture 204 into anterior lens element 194 and to refracting surface 208 where it exits the lens and proceeds approximately 41.5 mm to the biomicroscope objective lens aperture.

The exemplary lens as described, comprising an anterior plus powered aspheric reflector paired with a posterior minus powered aspheric reflector formed as an annulus defining a central aperture that transmits light directly through the lens, each which respectively produce the stated posterior and negative and anterior and negative reflections, provides a two-element optical system for a diagnostic and therapeutic gonioscopy lens that facilitates positioning of the lens on an examined eye and provides a real inverted image of the anterior chamber structures while simultaneously allowing direct viewing and diagnosis of other structures of the eye through the clear central viewing aperture.

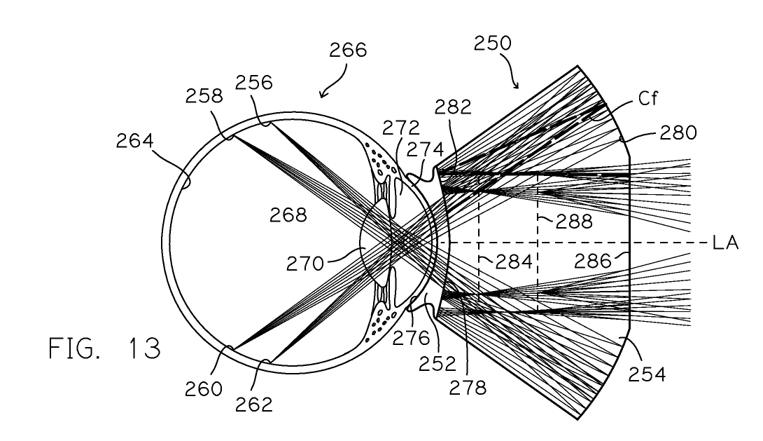

Referring to FIG. 12, there is shown a ray tracing and schematic cross-sectional view of an exemplary triplet gonioscopy lens according to an eighth embodiment of the invention, wherein lens 220 comprises an optically coupled lens including posterior contacting element 222, middle element 224 and anterior element 226. In this embodiment anterior lens element 226 is a spherical meniscus lens that both covers and protects the anterior reflecting surface and provides increased magnification of the inverted real image. The posterior reflecting surface is formed as an annulus in which the non-reflective central area or portion provides transmission of light directly through the lens. Posterior element 222 and middle element 224 are made of optical quality polycarbonate and anterior element 226 is made of N-BK7 glass. As a cemented triplet, the elements 222 and 224 may be adhered together at their interface using optical adhesive OP-4-20658 and elements 224 and 226 may be adhered together at their interface using 984 optical adhesive manufactured by Dymax Corporation.

Referring to FIG. 12, light beams 2g and 3g emanating from the stated iridocorneal and peripheral iris locations of anterior chamber 4g of eye 6g pass through the cornea 8g and tear layer of the eye and enter posterior contacting element 222 of lens 220 through corneal contacting surface 228 and continue through interface 230, comprised of the anterior and posterior surfaces of lens elements 222 and 224 respectively, optically coupled with an interface material. The light beams continue to concave annular reflecting surface 232 from which each light beam is first reflected as a negative reflection in a posterior direction. The convergent light beams proceed in their respective directions to convex annular reflecting surface 234 defined by inner diameter aperture 236 and refracting interface area 230, from which each light beam is next reflected as a negative reflection in an anterior direction. Proceeding from annular reflecting surface 234 the light beams focus at real inverted image 238 and continue through interface 240, comprised of the anterior and posterior surfaces of lens elements 224 and 226 respectively, optically coupled with an interface material. The light beams proceed through anterior lens element 226 in their respective directions towards surface 242 where they are refracted and exit the lens. Virtual image 244, which is the apparent location of real image 238, is located 14.3 mm posterior of surface 242. Anterior element 226 provides added magnification resulting in a virtual image magnification increased over that of the real image by a factor of approximately 1.49. The span of beam 2g 100 mm anterior of virtual image 244 exceeds a 30 mm diameter centered about lens axis LA.

Contacting surface 228 comprises a concave surface adapted for placement on the patient's cornea, and may have a spherical or aspherical curvature. In the exemplary lens of this embodiment surface 228 has a radius of 8.0 mm and is spherical. Optical interface 230 is the interface of the peripheral and central refracting portions of the anterior and posterior surfaces respectively of lens elements 222 and 224 and includes aperture 236. The peripheral refracting portion of interface 230 is plano. Reflecting surface 232 has a concave aspheric curvature with an apical radius of 18.5 mm and together with the anterior surface of element 224 within interface 240 comprises a surface of continuous curvature as the anterior surface of lens element 224. Reflecting surface 232 comprises an internally reflecting mirror-coated annular section having a 20 mm inner diameter that surrounds refracting surface area 240. Annular reflecting surface 234 has a convex aspheric curvature with an apical radius of 5.54 mm and together with aperture 236 and plano refracting surface area 230 comprises a lenticulated surface as the posterior surface of lens element 224. Reflecting surface 234 also comprises an internally reflecting mirror-coated annular section having a 7.1 mm outer diameter and a 4.2 mm inner diameter that surrounds refracting aperture 236. Aperture 236 has a diameter of 4.2 mm and is the clear non-reflective central area or portion through posterior annular reflector 234. Alternatively, the area of aperture 236 or an area along the lens axis proximate aperture 236 comprising the non-reflective portion may be non-transmissive of light including that from the illumination portion of a slit lamp biomicroscope. Optical interface 240 comprises the generally matched but slightly differently shaped anterior and posterior surfaces respectively of lens elements 224 and 226. The anterior refracting portion of lens element 224 of optical interface 240 has a convex aspheric curvature with an apical radius of 18.5 mm. The posterior surface of lens element 226 is concave and spherical with a radius of 18.34 mm. The slight curvature difference of each results in a thickness deviation over the extent of the interface medium of interface 240 of less than 0.02 mm. Anterior surface 242 of lens element 226 is convex and spherical with a radius of 17.3 mm.

Also shown in FIG. 12 is an alternative illumination arrangement using a series of very small LEDs embedded in the portion of the lens adjacent the cornea, angled to direct illumination at an angle across the anterior chamber to an opposing side of the iris and iridocorneal angle, thereby providing continuous illumination of anterior chamber structures even with movement of the biomicroscope during image scanning, LED 246 represents one of a series of embedded LEDs in the lid flange portion of lens element 222 forming a ring arrangement centered around lens axis LA and directing emitted light at an angle of approximately 60° with respect to lens axis LA to illuminate the anterior chamber structures.

Referring to FIG. 13, there is shown a ray tracing and schematic cross-sectional view of an exemplary doublet indirect opthalmoscopy contact lens according to a ninth embodiment of the invention, wherein lens 250 comprises an optically coupled lens including posterior element 252 and anterior element 254. The lens receives light rays from points in the mid-peripheral fundus and through refraction and reflection means similar to that of prior embodiments focuses the rays to form an inverted real image as a continuous annular section anterior of the examined eye. In this embodiment the anterior surface of lens element 254 comprises a lenticulated surface and both the anterior and posterior reflecting surfaces are aspheric. Posterior element 252 is made of optical quality polymethylmethacrylate and anterior element 254 is made of optical quality polycarbonate. As a cemented doublet, the two elements 252 and 254 may be adhered together at their interface using a suitable optical adhesive such as 3-20261.

Referring to FIG. 13, light beams 256, 258, 260 and 262 emanating from points on the retina 264 of eye 266 pass through the vitreous humor 268, crystalline lens 270, anterior chamber 272, cornea 274 and tear layer of the eye and enter posterior lens element 252 of lens 250 through contacting surface 276 and continue through interface 278, comprised of the anterior and posterior surfaces of lens elements 252 and 254 respectively, optically coupled with an interface material.

The light beams continue to concave annular reflecting surface 280 from which each light beam is first reflected as a positive reflection in a posterior direction. The convergent light beams proceed in their respective directions to convex annular reflecting surface 282 from which each light beam is next reflected as a negative reflection in an anterior direction. Proceeding from reflecting surface 282 the light beams focus at real inverted image 284 and continue in their respective directions towards surface 286 where they are refracted and exit the lens. Virtual image 288, which is the apparent location of real image 284, is located 9.27 mm posterior of surface 286. With respect to light beam 282, the value for the combined angles of reflection of central ray Cf from reflecting surfaces 280 and 282 is approximately 22° and the angle formed between central ray Cf and lens axis LA after its reflection from surface 282 is approximately 2.2°.

Contacting surface 276 comprises a concave surface adapted for placement on the patient's cornea, and may have a spherical or aspherical curvature. In the exemplary lens of this embodiment surface 276 has an apical radius of 7.7 mm and is aspheric. Optical interface 278 is the interface of the central refracting portions of the anterior and posterior surfaces respectively of lens elements 252 and 254. The curvature of interface 278 with respect to lens element 254 is aspheric and concave with an apical radius of 19.0 mm. Reflecting surface 280 has a concave aspheric curvature with an apical radius of 25.0 mm and together with refracting surface 286 comprises a lenticulated surface as the anterior surface of lens element 254. Refracting surface 286 is plano. Reflecting surface 280 comprises an internally reflecting mirror-coated annular section having a 16 mm inner diameter that surrounds refracting surface area 286. Annular reflecting surface 282 has a convex aspheric curvature with an apical radius of 19.0 mm and together with refracting surface area 278 comprises a continuous surface as the posterior surface of lens element 254. Reflecting surface 282 also comprises an internally reflecting mirror-coated annular section having a 10.4 mm inner diameter that surrounds refracting surface area 278. The reflective sections may be mirrored by means previously mentioned.

The exemplary lens as shown and described with reference to FIG. 13, comprising a first anterior plus powered aspheric reflector paired with a second posterior minus powered aspheric reflector, each which respectively produce the first stated posterior and positive and anterior and negative reflections, provides a two-element optical system for a diagnostic and therapeutic indirect opthalmoscopy contact lens with excellent imaging qualities providing a mid peripheral view of the fundus of the eye. It should be understood that other designs and embodiments of indirect opthalmoscopy contact lenses following the basic precepts of the present disclosure as outlined and described with respect to the various gonioscopy lens embodiments herein depicted are within the scope of the invention and therefore to avoid repetition these designs and embodiments are not herein included.

The invention has been described in detail with respect to various embodiments and it will now be apparent from the foregoing to those skilled in the art that changes and modifications may be made without departing from the invention in its broader aspects. For example, the embodiments describing lenses of the present disclosure made of particular glass or plastic materials may instead be made with any other polymer or other optical glass having any refractive index and Abbe value. It should be further understood that materials such as high temperature polymers suitable for optical applications may be used as replacements for acrylic or polycarbonate in order to accommodate high temperature sterilization procedures. As a further modification, additional lens elements may be incorporated into any of the embodiment designs without departing from the scope of the invention. Furthermore, any of the embodiments may incorporate a transparent or light filtering glass or plastic protective cover, and any refracting surfaces may be coated with an anti-reflective coating to lessen glaring reflection. It should be further understood that surfaces of lens embodiments using spherical curvatures may instead use aspheric curvatures and visa versa and that a lens design may be specifically adapted for use based on the particular design of the biomicroscope or other instrument used to capture the light rays as well as the refractive status of the examined eye. It should be further understood that lenses of any of the embodiments may be provided with a centrally positioned light stop to prevent visualization and/or slit lamp illumination of the retina or laser energy entering the posterior chamber. It should be further understood that the illumination source may be other than that of a standard full wavelength white light illumination source, for example, the illumination may comprise light of limited or monochromatic wavelengths, ultraviolet or infrared wavelengths, or may comprise a laser or scanning laser, and that an image capture system used in conjunction with the lens may utilize such monochromatic or laser or laser scanned light. The invention, therefore, as defined in the appended claims is intended to cover all such changes and modifications as fall within the true spirit of the invention.

What is claimed is:

1. An inverted real image forming opthalmoscopic contact lens for viewing or treating a structure within an eye, comprising:
a lens axis;
a contacting surface adapted for placement on a cornea of an eye, wherein the eye includes an anterior chamber and a posterior chamber;
a concave annular anterior reflecting surface positioned anterior of the contacting surface;
a convex annular posterior reflecting surface positioned posterior of the concave annular anterior reflecting surface;
a first non-reflective portion positioned along the lens axis and proximate to the convex annular posterior reflecting surface; and
a second non-reflective portion positioned along the lens axis and proximate to the concave annular anterior reflecting surface;
wherein a central ray of a light beam emanating from the structure within the eye, entering the lens through the contacting surface and contributing to the formation of the inverted real image is reflected within the lens in an ordered sequence of reflections, first in a posterior direction by the concave annular anterior reflecting surface and next as a negative reflection in an anterior direction by the convex annular posterior reflecting surface.

2. The opthalmoscopic contact lens of claim 1, wherein the inverted real image is the final real image formed by the lens.

3. The opthalmoscopic contact lens of claim 2, wherein the first reflection in the ordered sequence of reflections is a positive reflection.

4. The opthalmoscopic contact lens of claim 3, wherein a combined value of the angle of reflection of the first reflection as a positive reflection in a posterior direction by the anterior reflecting surface and the angle of reflection of the next reflection as a negative reflection in an anterior direction by the posterior reflecting surface is less than 24.5°.

5. The opthalmoscopic contact lens of claim 4, wherein a combined value of the angle of reflection of the first reflection as a positive reflection in a posterior direction by the anterior reflecting surface and the angle of reflection of the next reflection as a negative reflection in an anterior direction by the posterior reflecting surface is less than 18.5°.

6. The opthalmoscopic contact lens of claim 4, wherein an angle formed between the central ray after the next reflection as a negative reflection in an anterior direction by the posterior reflecting surface and the lens axis is less than 15°.

7. The opthalmoscopic contact lens of claim 4, wherein an angle formed between the central ray after the next reflection as a negative reflection in an anterior direction by the posterior reflecting surface and the lens axis is less than 8°.

8. The opthalmoscopic contact lens of claim 6, further comprising a refracting surface positioned anterior of the concave annular posterior reflecting surface.

9. The opthalmoscopic contact lens of claim 8, wherein the light beam is refracted through the refracting surface;
further wherein a span of the light beam centered about the lens axis at a distance 100 millimeters anterior of an apparent location of the inverted real image is at least 30 millimeters.

10. The opthalmoscopic contact lens of claim 9, wherein the structure is a structure within the anterior chamber of the eye.

11. The opthalmoscopic contact lens of claim 10, further comprising a substantially transparent path through the opthalmoscopic contact lens generally along the lens axis and passing through the first non-reflective portion and the second non-reflective portion;
wherein a second light beam emanating from a second structure of the eye, entering the lens through the contacting surface, passing through the transparent section and exiting the lens through the refracting surface contributes to the formation of a virtual image of the second structure of the eye.

12. The opthalmoscopic contact lens of claim 11, wherein the second structure is selected from the group consisting of a structure of the cornea, a structure of the iris, a structure of the lens capsule, and a structure of the fundus.

13. The opthalmoscopic contact lens of claim 11, wherein the lens is a doublet lens including a posterior element and an anterior element, further wherein one of the concave annular anterior reflecting surface and the convex annular posterior reflecting surface comprises a mirrored surface which is externally reflecting.

14. The opthalmoscopic contact lens of claim 11, wherein the lens is a triplet lens including a posterior element, a middle element and an anterior element, further wherein at least one of the concave annular anterior reflecting surface and the convex annular posterior reflecting surface comprises a mirrored surface which is externally reflecting.

15. The opthalmoscopic contact lens of claim 11, wherein the lens is a doublet lens including a posterior element and an anterior element;
further wherein the convex annular posterior reflecting surface has increasing curvature.

16. The opthalmoscopic contact lens of claim 11, wherein the lens is a triplet lens including a posterior element, a middle element and an anterior element;
further wherein the convex annular posterior reflecting surface has increasing curvature.

17. The opthalmoscopic contact lens of claim 6, further comprising an image sensor for converting light contributing to the formation of the inverted real image to an electrical signal.

18. The opthalmoscopic contact lens of claim 11, further comprising a light source for illuminating an area including the structure.

19. The opthalmoscopic contact lens of claim 18, wherein the light source comprises a plurality of light emitting diodes positioned in a ring formation in a location selected from the group consisting of a location anterior the convex annular posterior reflecting surface and a location posterior the convex annular posterior reflecting surface.

20. The opthalmoscopic contact lens of claim 19, wherein the location of the plurality of light emitting diodes is a location posterior the convex annular posterior reflecting surface;
further wherein the plurality of light emitting diodes is at least partially embedded in the lens.

21. The opthalmoscopic contact lens of claim 19, further comprising an electric cell for powering the plurality of light emitting diodes.

22. The opthalmoscopic contact lens of claim 21, further comprising a compartment for containing the electric cell located anterior the concave annular anterior reflecting surface.

23. The opthalmoscopic contact lens of claim 11, further comprising a fiber optic light guide for directing emitted light to illuminate an area including the structure.

24. The opthalmoscopic contact lens of claim 23, wherein the fiber optic light guide contacts a surface of the lens or enters a portion of the lens adjacent the contacting surface.

25. The opthalmoscopic contact lens of claim 11, wherein the concave annular anterior reflecting surface is defined by a first prescription and the refracting surface is defined by a second prescription that is different than the first prescription;
further wherein the concave annular anterior reflecting surface and the refracting surface join tangentially and without discontinuity.

26. The opthalmoscopic contact lens of claim 11, wherein the concave annular anterior reflecting surface is defined by a first prescription and a second refracting surface is defined by a second prescription that is different than the first prescription;
further wherein the concave annular anterior reflecting surface and the second refracting surface join tangentially and without discontinuity.

27. The opthalmoscopic contact lens of claim 11, wherein the concave annular anterior reflecting surface and the refracting surface comprise a lenticular surface.

28. The opthalmoscopic contact lens of claim 8, wherein the structure is a structure within the posterior chamber of the eye.

29. The opthalmoscopic contact lens of claim 2, wherein the first reflection in the ordered sequence of reflections is a negative reflection.

30. The opthalmoscopic contact lens of claim 29, further comprising a refracting surface positioned anterior of the concave annular posterior reflecting surface.

31. The opthalmoscopic contact lens of claim 30, wherein the light beam is refracted through the refracting surface;
further wherein a span of the light beam centered about the lens axis at a distance of 100 millimeters anterior of an apparent location of the inverted real image is at least 30 millimeters.

32. The opthalmoscopic contact lens of claim 31, wherein the structure is a structure within the anterior chamber of the eye.

33. The opthalmoscopic contact lens of claim 32, further comprising a substantially transparent path through the opthalmoscopic contact lens generally along the lens axis and through the first non-reflective portion and the second non-reflective portion;
wherein a second light beam emanating from a second structure of the eye, entering the lens through the contacting surface, passing through the transparent section and exiting the lens through the refracting surface contributes to the formation of a virtual image of the second structure of the eye.

34. The opthalmoscopic contact lens of claim 33, wherein the second structure is selected from the group consisting of a structure of the cornea, a structure of the iris, a structure of the lens capsule, and a structure of the fundus.

35. The opthalmoscopic contact lens of claim 33, wherein the lens is a doublet lens including a posterior element and an anterior element;
further wherein the vertex of a curvature defining the refracting surface is displaced in an anterior direction from the vertex of a curvature defining the concave annular anterior reflecting surface.

36. The opthalmoscopic contact lens of claim 35, wherein refraction of the light beam by the refracting surface provides magnification of the inverted real image by a factor of at least 1.4.

37. The opthalmoscopic contact lens of claim 33, wherein the lens is triplet lens including a posterior element, a middle element and an anterior element;
further wherein refraction of the light beam by the refracting surface provides magnification of the inverted real image by a factor of at least 1.4.

38. The opthalmoscopic contact lens of claim 37, wherein a diameter of the anterior element is substantially equal to a diameter of the concave annular anterior reflecting surface.

39. The opthalmoscopic contact lens of claim 33, wherein the convex annular posterior reflecting surface has increasing curvature.

40. The opthalmoscopic contact lens of claim 32, wherein the first non-reflective portion is non-transmissive to at least one wavelength of light.

41. The opthalmoscopic contact lens of claim 29, further comprising an image sensor for converting light contributing to the formation of the inverted real image to an electrical signal.

42. The opthalmoscopic contact lens of claim 40, further comprising light emitting diodes for directing emitted light to illuminate an area including the structure.

43. The opthalmoscopic contact lens of claim 33, further comprising a light source for illuminating an area including the structure.

44. The opthalmoscopic contact lens of claim 43, wherein the light source comprises a plurality of light emitting diodes positioned in a ring formation in a location selected from the group consisting of a location anterior the convex annular posterior reflecting surface and a location posterior the convex annular posterior reflecting surface.

45. The opthalmoscopic contact lens of claim 44, wherein the location is a location posterior the convex annular posterior reflecting surface;
further wherein the plurality of light emitting diodes is at least partially embedded in the lens.

46. The opthalmoscopic contact lens of claim 44, further comprising an electric cell for powering the plurality of light emitting diodes.

47. The opthalmoscopic contact lens of claim 46, further comprising a compartment for containing the electric cell located anterior the concave annular anterior reflecting surface.

48. The opthalmoscopic contact lens of claim 33, further comprising a fiber optic light guide for directing emitted light to illuminate an area including the structure.

49. The opthalmoscopic contact lens of claim 48, wherein the fiber optic light guide contacts a surface of the lens or enters a portion of the lens adjacent the contacting surface.

50. A method for manufacturing an inverted real image forming opthalmoscopic contact lens having axial symmetry, comprising:
forming a contacting surface adapted for placement on a cornea of an eye including an anterior chamber and a posterior chamber and further adapted to permit entrance into the lens of a central ray of a light beam emanating from a structure within the eye and contributing to the formation of the inverted real image of the structure;
forming a concave annular anterior reflecting surface positioned anterior of the contacting surface and adapted to reflect the central ray in a posterior direction that is a first reflection in an ordered sequence of reflections;
forming a first non-reflective portion positioned along an axis of symmetry and proximate to the concave annular anterior reflecting surface;
forming a convex annular posterior reflecting surface positioned posterior of the concave anterior reflecting surface and adapted to reflect the central ray in an anterior direction as a negative reflection that is a next reflection in the ordered sequence of reflections; and
forming a second non-reflective portion positioned along the axis of symmetry and proximate to the convex annular posterior reflecting surface;
wherein the inverted real image is the final real image formed by the lens.

51. The method of claim 50, wherein the concave annular anterior reflecting surface is adapted to reflect the central ray as a positive reflection that is the first reflection in the ordered sequence of reflections.

52. The method of claim 51, wherein the concave annular anterior reflecting surface is adapted to reflect the central ray that is the first reflection at a first angle of reflection, and the convex annular posterior surface is adapted to reflect the central ray that is the next reflection at a second angle of reflection;
further wherein a combined value of the first angle of reflection and the second angle of reflection is less than 24.5°.

53. The method of claim 52, wherein the convex annular posterior reflecting surface is adapted to reflect the central ray as the negative reflection in an anterior direction that is the next reflection from the convex annular posterior reflecting surface at an angle with the axis of symmetry that is less than 15°.

54. The method of claim 53, further comprising forming a refracting surface;
wherein the refracting surface is positioned anterior of the convex annular posterior reflecting surface.

55. The method of claim 54, wherein the refracting surface is adapted to refract the light beam to span a 30 millimeter extent centered about the axis of symmetry at a distance 100 millimeters anterior of an apparent location of the inverted real image.

56. The method of claim 55, wherein the concave annular anterior reflecting surface is adapted to form a first substantially transparent section through the opthalmoscopic contact lens along the axis of symmetry inward the concave annular anterior reflecting surface, and the convex annular posterior reflecting surface is adapted to form a second substantially transparent section through the opthalmoscopic contact lens along the axis of symmetry inward the convex annular posterior reflecting surface;
further wherein the contacting surface is adapted to permit entrance into the lens of a second light beam emanating from a second structure of the eye to pass through the first and second substantially transparent sections, and the refracting surface is adapted to permit refraction of the second light beam to exit the lens contributing to the formation of a virtual image of the second structure of the eye.

57. The method of claim 56, further comprising forming a plurality of light emitting diodes;
wherein the plurality of light emitting diodes is adapted to permit illumination of an area including the structure.

58. The method of claim 57, wherein the plurality of light emitting diodes is formed at least partially embedded in the lens.

59. The method of claim 58, further comprising forming a compartment for containing an electric cell for powering the plurality of light emitting diodes;
wherein the compartment is positioned anterior to the concave annular anterior reflecting surface.

60. The method of claim 56, further comprising forming a fiber optic light guide positioned adjacent the lens;
wherein the fiber optic light guide is adapted for directing emitted light to illuminate an area including the structure.

61. The method of claim 50, wherein the concave annular anterior reflecting surface is adapted to reflect the central ray as a negative reflection that is the first reflection in the ordered sequence of reflections.

62. The method of claim 61, further comprising forming a refracting surface;
wherein the refracting surface is positioned anterior of the concave annular posterior reflecting surface.

63. The method of claim 62, wherein the refracting surface is adapted to refract the light beam to span a 30 millimeter extent centered about the axis of symmetry at a distance 100 millimeters anterior of an apparent location of the inverted real image.

64. The method of claim 63, wherein the contacting surface, the concave annular anterior reflecting surface, the convex annular posterior reflecting surface, and the refracting surface are adapted to form the inverted real image of the structure that is a structure within the anterior chamber of the eye.

65. The method of claim 64, wherein the concave annular anterior reflecting surface is adapted to form a first substantially transparent section through the opthalmoscopic contact lens along the axis of symmetry inward the concave annular anterior reflecting surface, and the convex annular posterior reflecting surface is adapted to form a second substantially transparent section through the opthalmoscopic contact lens along the axis of symmetry inward the convex annular posterior reflecting surface;
further wherein the contacting surface is adapted to permit entrance into the lens of a second light beam emanating from a second structure of the eye to pass through the first and second substantially transparent sections, and the refracting surface is adapted to permit refraction of the second light beam to exit the lens contributing to the formation of a virtual image of the second structure of the eye.

66. The method of claim 65, wherein the refracting surface is adapted to refract the light beam to provide magnification of the inverted real image by a factor of at least 1.4.

67. The method of claim 64, wherein the convex annular posterior reflecting surface is adapted to form a non-transmissive section along the axis of symmetry inward the convex annular posterior reflecting surface;
further wherein the non-transmissive section is adapted to prevent transmission of at least one wavelength of light.

68. The method of claim 65, further comprising forming a plurality of light emitting diodes;
wherein the light emitting diodes are adapted to permit illumination of an area including the structure.

69. The method of claim 68, wherein the plurality of light emitting diodes is formed at least partially embedded in the lens.

70. The method of claim 68, further comprising forming a compartment for containing an electric cell for powering the plurality of light emitting diodes;
wherein the compartment is positioned anterior the concave annular anterior reflecting surface.

71. The method of claim 67, further comprising forming a plurality of light emitting diodes;
wherein the plurality of light emitting diodes is adapted to permit illumination of an area including the structure.

72. The method of claim 65, further comprising forming a fiber optic light guide positioned adjacent the lens;
wherein the fiber optic light guide is adapted for directing emitted light to illuminate an area including the structure.
